(19) United States
(12) Patent Application Publication
(10) Pub. No.: US 2023/0398221 A1
(43) Pub. Date: Dec. 14, 2023

(54) SYSTEM FOR FOCUSED TARGETING OF MAGNETO-AEROTACTIC-RESPONSIVE BACTERIA AND METHOD OF USE THEREOF

(71) Applicant: STARPAX BIOPHARMA INC., Montréal (CA)

(72) Inventor: Sylvain Martel, île Bizard (CA)

(21) Appl. No.: 18/333,958

(22) Filed: Jun. 13, 2023

Related U.S. Application Data
(60) Provisional application No. 63/351,938, filed on Jun. 14, 2022.
(60) Provisional application No. 63/351,950, filed on Jun. 14, 2022.

(51) Int. Cl. A61K 41/00; A61K 9/00; A61K 49/18; A61K 47/69

(52) U.S. Cl. CPC A61K 41/00; A61K 9/0009; A61K 49/1896; A61K 47/6901

(57) ABSTRACT

A method of improving targeted delivery of at least one of a treatment agent, an imaging agent and a diagnostic agent attached to magneto-aerotactic-responsive bacteria; it includes adapting a total bolus escape time of a solution of the magneto-aerotactic responsive bacteria in order to influence the targeting of a target zone with hypoxic zones in the patient.

The present disclosure claims priority from U.S. provisional patent application No. 63/351,938, filed on Jun. 14, 2022, incorporated herein by reference, and U.S. provisional patent application No. 63/351,950, filed on Jun. 14, 2022, incorporated herein by reference.

### TECHNICAL FIELD

The present disclosure relates to magnetotaxis, and more particularly to navigating magneto-aerotactic-responsive bacteria using a magnetic field for imaging, diagnosis, and/or therapy.

### BACKGROUND

Use of a magnetic field for medical interventions is becoming increasingly more prevalent. For instance, the following patent documents describe prior art medical applications harnessing a magnetic field: U.S. Pat. Nos. 9,655,539, 9,381,063, 9,220,425, 8,986,214, 8,684,010, 8,457,714, US20130006100, US20120310111, US20120289822, US20120288838, US20110092808, U.S. Pat. No. 7,873,401, US20100305402, US20090275828, US20090248014, US20050096589, etc.

Recently, the use of magnetotactic entities, used with an applied magnetic field, has also been studied for medical purposes.

Magnetotactic entities are defined as untethered entities where the source of propulsion or the system responsible for the displacement of the entity is part of, attached to, or embedded in the entity itself. Magnetotactic entities include a group of objects or microorganisms and any biological system or hybrid system including micro- and nano-systems or structures made of biological and/or synthetic (including chemical, artificial, etc.) materials and/or components where the directional motion can be influenced by inducing a torque from a directional magnetic field (e.g. from a permanent magnet) or electro-magnetic field (magnetic field includes here electro-magnetic field generated by an electrical current flowing in a conductor), a method referred to here as magnetotaxis where the direction of motion of such magnetotactic entities is influenced by a directional magnetic field (the magnetotactic entities can also be functionalized and be attached to other structures if required). Examples of such magnetotactic entities include but are not limited to a single or a group (swarm, agglomeration, aggregate, etc.) of flagellated Magnetotactic Bacteria (MTB), or other bacteria or other microorganisms capable of self-propulsion and influenced for the purpose of directional control by a directional magnetic field that could have been modified previously accordingly from various methods including but not limited to cultivation parameters, genetics, or attached, embedded to other entities modified to allow control by magnetotaxis such as other cells (including red blood cells), or attached to a synthetic structure that can be influenced by a directional magnetic field, or by adding micro- or nano-components to the bacteria, cells, or other microorganisms to make the directional motion of the implementation including hybrid (made of biological and synthetic components) implementation sensitive to magnetotaxis or a directional magnetic field such as the one capable of influencing the direction of a magnetic nano-compass needle.

However, due to the environment resulting from the human body, such as body temperature, pH, concentrations of solutes, etc., this environment, hostile to the bacteria once introduced into the subject, reduces the activity and motility of the magnetotactic bacteria following their injection therein. As such, there remains a need for establishing protocols for improving targeting of the magnetotactic bacteria for allowing the magnetotactic bacteria to reach a target zone prior to the magnetotactic bacteria losing their motility, or perishing.

### SUMMARY

The present disclosure relates to methods for modulating a magnetic field used for steering magneto-aerotactic-responsive bacteria for treatment, diagnosis and/or imaging of a subject.

The methods employ the aerotactic and magnetotactic properties of the magneto-aerotactic-responsive bacteria. The magneto-aerotactic-responsive bacteria are adapted to follow an oxygen gradient from high oxygen to low oxygen and are adapted to be steered by a magnetic field. This affinity for a decreasing concentration of oxygen results in the magneto-aerotactic-responsive bacteria being drawn to hypoxic zones in the subject (e.g. such as a tumor, but not limited thereto), where the hypoxic zones have little oxygen. The magneto-aerotactic-responsive bacteria are able to self-locomote to the hypoxic zones of the target zone using their self-propelling system (e.g. their flagella). This may occur with or without the presence of a magnetic field once the magneto-aerotactic-responsive bacteria are in the presence of an oxygen gradient, where relaxation time of run-and-tumble motion is increased, resulting in change of direction of movement of the bacteria from the direction of the magnetic field.

Therefore, the present method pertains to modulating the magnetic field intensity of a magnetic field in order to change the movement pattern of the bacteria as a function of the intensity of the magnetic field. A greater magnetic field intensity favours a run-and-reverse motion—causing displacement of the bacteria—as the bacteria follows the magnetic field. At lower magnetic field intensities, the magneto-aerotactic-responsive bacteria adopt more of a run-and-tumble motion as they seek out oxygen gradients, leading the magneto-aerotactic-responsive bacteria to hypoxic zones in the subject.

The present method leverages changes in the types of movement of the magneto-aerotactic-responsive bacteria as a function of environmental factors (e.g. magnetic field intensity) to improve targeting of the magneto-aerotactic-responsive bacteria in the patient. By modulating the magnetic field intensity, the movement of the bacteria through run-and-tumble can be controlled, where a higher magnetic field intensity reduces changes in direction of movement of the bacteria through run-and-tumble, and a lower magnetic field intensity increases changes in direction of movement through run-and-tumble motion, thereby allowing the bacteria to locate the hypoxic zones.

Run-and-reverse motion can be expressed as bacteria typically exhibiting long unidirectional runs being interrupted by short reversal events with a specific mean aperiodic frequency, resulting in forward displacements. In contrast, run-and-tumble motion can be characterized by perturbations in an alignment between a direction of motion of the magneto-aerotactic-responsive bacterium with the direction of the magnetic field due to tumbles (where the bacteria undergo a change of direction of motion from the direction of the magnetic field), followed by relaxation of the alignment with the directional magnetic field during the run state. If the run state time is shorter than the relaxation time, the magnetic field does not have sufficient time to realign the magneto-aerotactic-responsive bacterium before the next tumble causing the direction of motion of the magneto-aerotactic-responsive bacterium cell to fluctuate from the direction of the magnetic field.

The methods described herein may employ two to three modes of navigating the magneto-aerotactic-responsive bacteria. In a first mode, the magnetic field intensity is set such that magnetotaxis dominates, where the noticeable motion of the bacteria is primarily run-and-reverse, the higher magnetic field intensity increasing the run state of the bacteria over the relaxation time during run-and-tumble. In a second mode, the motion of the bacteria may be caused by both magnetotaxis and aerotaxis by lowering the magnetic field intensity, where the bacteria exhibit more changes in direction resulting from run-and-tumble motion. In a third mode, where the magnetic field intensity is even lower than in the second mode, the motion of the bacteria may be caused primarily by aerotaxis, where the bacteria exhibit primarily changes of direction from run-and-tumble motion.

It will be understood that one mode may be used more than once when navigating the bacteria, and that the order of the modes may vary depending on the given trajectory of the bacteria, the properties of the target zone, the location of the target zone, etc. A user may select one or more modes, in varying sequences, in order for reaching the target zone and the desired population of hypoxic zones.

The run-and-reverse motion enables the magneto-aerotactic-responsive bacteria to move to a different location in the subject (e.g. used primarily for displacement over greater distances), where the run-and-tumble motion improves the seeking of the magneto-aerotactic-responsive bacteria of the hypoxic zones. The magnetic field intensity can be adapted as a function of the desired movement pattern of the magneto-aerotactic-responsive bacteria, the properties of the target in the subject (e.g. density of tissue; size; concentration of hypoxic zones, etc.), the distribution of velocity pre-injection, the regression in velocity of the bacteria post-injection, the volume and dimensions of the bolus, the concentration of the magneto-aerotactic-responsive bacteria in the bolus, the distance between the injection site and the target, etc.

In some embodiments, as the speed of the magneto-aerotactic-responsive bacteria is greater when the magneto-aerotactic-responsive bacteria are directed by the magnetic field at the higher magnetic field intensity, this higher magnetic field intensity is maintained to navigate the magneto-aerotactic-responsive bacteria until the magneto-aerotactic-responsive bacteria reach the target zone with one or more oxygen gradients (e.g. resulting from one or more hypoxic zones). Once the magneto-aerotactic-responsive bacteria reach the target zone, the lowering of the magnetic field intensity of the oxygen gradient causes a change in the movement pattern of the magneto-aerotactic-responsive bacteria. With this different movement pattern, the magneto-aerotactic-responsive bacteria follow a run-and-tumble movement, resulting in changes in direction of movement of the magneto-aerotactic-responsive bacteria. These changes in direction of the magneto-aerotactic-responsive bacteria permit the magneto-aerotactic-responsive bacteria to locate the hypoxic zones, where the magneto-aerotactic-responsive bacteria may remain until their velocity post-injection reduces to 0 (e.g. resulting in the deposit of an agent that is attached to the magneto-aerotactic-responsive bacteria).

As such, when the magneto-aerotactic-responsive bacteria are attached to a diagnostic agent, imaging agent and/or therapeutic agent, the magneto-aerotactic-responsive bacteria will immobilize (e.g. perish) in these hypoxic zones of the tumor, thereby leaving the diagnostic agent, imaging agent and/or therapeutic agent at the site of the hypoxic zone. In fact, as the diagnostic agent, imaging agent and/or therapeutic agent provides targeted delivery of the agents, this results in a lower dosage of the agent being required for delivery to the subject. This reduces toxicity for the subject.

In some embodiments, the velocity distribution of the magneto-aerotactic-responsive bacteria in a bolus or sample can be used to predict the behavior of the magneto-aerotactic-responsive bacteria once injected into the subject and exposed to a magnetic field. For instance, a narrower velocity distribution promotes a smaller spread (and smaller targeting volume) in the magneto-aerotactic-responsive bacteria once injected into the patient and influenced by the magnetic field, as the magneto-aerotactic-responsive bacteria have similar starting velocities. However, a broader velocity distribution would result in a greater spread or distance (and greater targeting volume) in the magneto-aerotactic-responsive bacteria over time after injection and exposure to the magnetic field, as some bacteria travel significantly slower than others when exposed to a magnetic field. A greater spread in the bacteria can cause a more diffuse targeting of hypoxic zones when the magnetic field intensity is modulated to increase the run-and-tumble motion for seeking out hypoxic zones. However, as the bacteria are spread across a greater number of hypoxic zones, their concentration per hypoxic zone will be reduced. In contrast, a narrower spread in the distance between the magneto-aerotactic-responsive bacteria post-injection (due to a smaller velocity distribution) would result in a more concentrated targeting of hypoxic zones when the magnetic field intensity is modulated to increase the run-and-tumble motion for seeking out hypoxic zones. This is because when the magnetic field intensity is decreased (favouring changes of direction of the bacteria from the direction of the magnetic field as a result of run-and-tumble motion (for seeking out hypoxic zones) increases), the bacteria are already in a smaller volume of the subject, where all of the injected bacteria have travelled close to the same distance from the injection site in the subject.

As a result, leveraging the velocity distribution of the magneto-aerotactic-responsive bacteria may be used (e.g. when preparing a solution for injection; when selecting a solution or sample for injection) to increase or decrease the number of hypoxic zones targeted, and the concentration of the magneto-aerotactic-responsive bacteria targeting each of the targeted hypoxic zones. If the magneto-aerotactic-responsive bacteria are to be diluted across a greater number of hypoxic zones, a sample with a greater velocity distribution may be selected or prepared. If the magneto-aerotactic-responsive bacteria are to be concentrated in a smaller number of hypoxic zones (e.g. less dilution), then a sample with a smaller velocity distribution may be selected or prepared.

In one embodiment, with regard to the magnetic field applied, the generated magnetic field provides a directional torque that can be defined in one or more of three axes through the use of three pairs of magnetic coils, where each pair of magnetic coils is arranged with respect to one axis. The pairs of magnetic heads can generate a 3D convergence point towards which the magnetotactic entities will navigate towards and converge. A three-dimensional convergence point (CP) in a magnetic field is a point, unbounded in space, to which the entities following the direction of the magnetic field in an aggregation zone (AZ) will move to and aggregate. The magnetic field at the convergence point is effectively zero, and surrounding the convergence point in the aggregation zone, the effective field points from all directions to the convergence point. Because a magnetic field is not a point source, at least one of the magnetic field sources will be time varied to cause the entities to move towards the convergence point and stay close to the convergence point.

Therefore, maintaining any two axes (x, y or z) with a constant (static) magnetic field and changing the direction of the other axis depending upon the other two axes being maintained constant will generate a convergence point. Similarly, maintaining one axis constant and changing the direction of the other two axes in a time multiplex-fashion at the same time (synchronized) or with a phase (delay) will function to generate a convergence point, provided that the change is done at a frequency that allows for appropriate reaction of the magneto-aerotactic-responsive bacteria (i.e. a low frequency, e.g. around 0.1 and 5 Hz or preferably about 0.5 Hz). The direction of all three axes can be changed in a time multiplexing fashion simultaneously or with a delay between each axis. All combinations are possible provided that the magnetic field gradient of at least one axis (x, y or z) changes direction in a time multiplexed fashion with a switching speed appropriate with the reaction time of the magneto-aerotactic-responsive bacteria. U.S. Pat. No. 9,905,347, incorporated herein by reference, describes a system for steering magnetotactic entities in a subject. U.S. Pat. No. 9,905,347 describes a system and method for generating a 3D-convergence point using at least three pairs of magnetic field sources arranged along three axes or in three planes.

A broad aspect is a method of obtaining at least one of imaging information, diagnosis and treatment of a subject using magneto-aerotactic-responsive bacteria adapted to self-locomote. The method includes obtaining imaging information of a target zone in the subject, whereby a bolus of magneto-aerotactic-responsive bacteria is injected into the subject, the magneto-aerotactic-responsive bacteria attached to at least one of a therapeutic agent, a diagnostic agent and an imaging agent; applying a magnetic field at a first magnetic field intensity to guide and cause a displacement of the magneto-aerotactic responsive bacteria towards the target zone having hypoxic zones through magnetotaxis; and applying a magnetic field with a second magnetic field intensity that is less than the first magnetic field intensity for allowing the magneto-aerotactic responsive bacteria to follow an oxygen gradient for drawing the bacteria to the hypoxic zones, wherein there is an increase in changes of direction of movement of the magneto-aerotactic responsive bacteria from a direction of the magnetic field at the second magnetic field intensity than at the first magnetic field intensity, for at least one of treatment, diagnosis and imaging of the subject.

In some embodiments, the method may include obtaining velocity distribution information of the magneto-aerotactic-responsive bacteria of the bolus to predict a spread post-injection of the magneto-aerotactic-responsive bacteria in the subject when the magnetic field intensity is at the first magnetic field intensity.

In some embodiments, the bolus may be selected from a plurality of magneto-aerotactic-responsive bacteria solutions as a function of obtained velocity distribution information pre-injection of the magneto-aerotactic-responsive bacteria in the magneto-aerotactic-responsive bacteria solutions, wherein the selected solution may include magneto-aerotactic-responsive bacteria with a velocity distribution that is greater as a function of a desired targeting volume with the magneto-aerotactic-responsive bacteria, wherein a greater targeting volume may allow for targeting of a greater number of hypoxic zones associated with the target zone.

In some embodiments, a solution, amongst the solutions, with the broadest velocity distribution pre-injection of the magneto-aerotactic-responsive bacteria may be selected when a greater targeting volume is sought for increasing the number of hypoxic zones targeted by the magneto-aerotactic-responsive bacteria.

In some embodiments, a solution, amongst the solutions, with the narrowest velocity distribution pre-injection of the magneto-aerotactic-responsive bacteria may be selected when concentrated targeting is sought for increasing the concentration of magneto-aerotactic-responsive bacteria targeting one or more hypoxic zones.

In some embodiments, the first magnetic field intensity and second magnetic field intensity may be determined by accounting for a regression of the velocity of the magneto-aerotactic-responsive bacteria post-injection.

In some embodiments, the method may include estimating a position of the magneto-aerotactic-responsive bacteria when the velocity of the magneto-aerotactic-responsive bacteria post-injection regresses to 0.

In some embodiments, the method may include calculating a distance between an injection site of the bolus and the aggregation zone or a portion or the tumor, wherein the applying a magnetic field with a second magnetic field intensity is based on the calculated distance.

In some embodiments, the magneto-aerotactic-responsive bacteria may be attached to an imaging agent, and wherein the imaging agent is a contrast agent.

In some embodiments, the contrast agent may be gadolinium.

In some embodiments, the obtaining imaging information of a tumor of a subject may be performed using an MRI or a CT scanner.

In some embodiments, the imaging information may include information on the arteries of the subject to avoid piercing the arteries when performing the injecting of magneto-aerotactic-responsive bacteria into a peripheral region of the tumor.

In some embodiments, the first magnetic field intensity and second magnetic field intensity may be determined and adjusted based on the volume of the bolus; a distance between an injection point of the bolus and the aggregation zone; and time that lapsed following the injecting.

In some embodiments, the first magnetic field intensity and second magnetic field intensity may be further determined and adjusted based on the concentration of magneto-aerotactic-responsive bacteria in the bolus.

In some embodiments, the magneto-aerotactic-responsive bacteria may be injected into a peripheral region of the tumor.

In some embodiments, a plurality of volumes may be injected into said subject at different sites on said subject.

In some embodiments, the method may include, after the reducing the magnetic field intensity of the magnetic field to a second magnetic field intensity, further reducing the magnetic field intensity of the magnetic field to a third magnetic field intensity that is less than the second magnetic field intensity, wherein the magneto-aerotactic-responsive bacteria may further exhibit an increase in changes of direction of movement of the magneto-aerotactic responsive bacteria from the direction of the magnetic field at the third magnetic field intensity than at the second magnetic field intensity.

In some embodiments, the second magnetic field intensity may be of a value of 0 Gauss or above, but less than 5 Gauss.

In some embodiments, the first magnetic field intensity may be at least 15 Gauss.

In some embodiments, the second magnetic field intensity may be less than 15 Gauss, but more than or equal to 5 Gauss.

In some embodiments, the second magnetic field intensity may be of a value of 0 Gauss or above, but less than 5 Gauss.

Another broad aspect is a system for at least one of obtaining imaging information, diagnosis and treatment of a subject using magneto-aerotactic-responsive bacteria adapted to self-locomote with run-and-reverse and run-and-tumble motions after an amount of magneto-aerotactic-responsive bacteria is injected into the subject, the magneto-aerotactic-responsive bacteria attached to at least one of a therapeutic agent, a diagnostic agent and an imaging agent. The system includes a processor; and memory storing program code that, when executed by the processor, cause the processor to obtain imaging information of a target zone in the subject; apply a magnetic field at a first magnetic field intensity to guide and cause a displacement of the magneto-aerotactic responsive bacteria towards the target zone having hypoxic zones through magnetotaxis; and apply a magnetic field with a second magnetic field intensity that is less than the first magnetic field intensity for allowing the magneto-aerotactic responsive bacteria to follow an oxygen gradient for drawing the bacteria to the hypoxic zones, wherein there is an increase in changes of direction of movement of the magneto-aerotactic responsive bacteria from a direction of the magnetic field at the second magnetic field intensity than at the first magnetic field intensity.

In some embodiments, the system may include a user input interface, wherein the program code further comprises instructions for causing the processor, when executing the program code, to receive instructions from the user inputted into the user input interface to generate the imaging information that is obtained.

In some embodiments, the system may include the one or more magnetic sources.

In some embodiments, the one or more magnetic sources may include three pairs of magnetic coils, wherein each pair of the three pairs of magnetic coils may be arranged with respect to one distinct axis of the three axes, x, y and z.

In some embodiments, the system may include the imaging device.

In some embodiments, the imaging device may be an MRI machine.

In some embodiments, the first magnetic field intensity may be at least 15 Gauss.

In some embodiments, the second magnetic field intensity may be less than 15 Gauss, but more than or equal to 5 Gauss.

In some embodiments, the program code for determining the first magnetic field intensity and second magnetic field intensity may consider the following the volume of the bolus; a distance between an injection point of the bolus and the aggregation zone; and time that lapsed following the injecting.

In some embodiments, the program code for determining the first magnetic field intensity and second magnetic field intensity may further consider the concentration of magneto-aerotactic-responsive bacteria in the bolus.

In some embodiments, the memory may include program code that, when executed by the processor, cause the processor to, after the reducing the magnetic field intensity of the magnetic field to the second magnetic field intensity, further reduce the magnetic field intensity of the magnetic field to a third magnetic field intensity that is less than the second magnetic field intensity, wherein the magneto-aerotactic-responsive bacteria further exhibit an increase in changes of direction of movement of the magneto-aerotactic responsive bacteria from the direction of the magnetic field at the third magnetic field intensity than at the second magnetic field intensity.

In some embodiments, the memory may include program code that, when executed by the processor, cause the processor to calculate a distance between an injection site of the bolus and the aggregation zone or a portion or the tumor, wherein the applying a magnetic field with a second magnetic field intensity accounts for the calculated distance.

In some embodiments, the memory may include program code that, when executed by the processor, cause the processor to estimate a position of the magneto-aerotactic-responsive bacteria when a velocity post-injection of the magneto-aerotactic-responsive bacteria regresses to 0.

Another broad aspect is non-transitory storage medium storing instructions executable by a computing device, comprising at least one instruction for obtaining imaging information of a target zone in the subject; at least one instruction for applying a magnetic field at a first magnetic field intensity to guide and cause a displacement of the magneto-aerotactic responsive bacteria towards the target zone in the subject having hypoxic zones; and applying a magnetic field with a second magnetic field intensity that is less than the first magnetic field intensity for allowing the magneto-aerotactic responsive bacteria to follow an oxygen gradient for drawing the bacteria to the hypoxic zones, wherein there is an increase in changes of direction of movement of the magneto-aerotactic responsive bacteria from a direction of the magnetic field at the second magnetic field intensity than at the first magnetic field intensity.

Another broad aspect is a method of selecting a sample of magneto-aerotactic-responsive bacteria attached to at least one of an imaging agent, targeting agent and diagnostic agent as a function of concentrated or diluted targeting of a target zone in a subject with hypoxic zones by the magneto-aerotactic-responsive bacteria that are steered using a magnetic field through magnetotaxis to the target zone in the subject and seek out hypoxic zones in the subject through aerotaxis. The method includes selecting a sample of magnetic-aerotactic-responsive bacteria amongst samples of magnetic-aerotactic-responsive bacteria as a function of a pre-injection velocity distribution of the magneto-aerotactic-responsive bacteria in the samples, wherein a sample with a greater pre-injection velocity distribution is selected as the targeting volume and number of target hypoxic zones is greater, and wherein a sample with a lesser pre-injection velocity distribution is selected when the targeting volume is lesser and the number of target hypoxic zones is lesser for more concentrated targeting by the magneto-aerotactic-responsive bacteria.

Another broad aspect is a method of preparing a solution of magneto-aerotactic-responsive bacteria for parenteral administration to a subject for achieving a sought level targeting volume at a target zone of the subject, wherein the administered magneto-aerotactic-responsive bacteria are steered using a magnetic field through magnetotaxis to the target zone in the subject and seek out hypoxic zones in the subject through aerotaxis. The method includes selecting magneto-aerotactic-responsive bacteria based on magnetotactic self-locomotive velocity of the magneto-aerotactic-responsive bacteria to obtain a population of selected magneto-aerotactic-responsive bacteria for the solution, wherein a velocity distribution of the selected magneto-aerotactic-responsive bacteria is greater as the targeting volume that is sought is greater; and preparing the solution including the selected magneto-aerotactic-responsive bacteria, wherein the selected magneto-aerotactic-responsive bacteria are attached to at least one of an imaging agent, a contrast agent and a diagnostic agent.

Another broad aspect is a method of improving targeted delivery of at least one of a treatment agent, an imaging agent and a diagnostic agent attached to magneto-aerotactic-responsive bacteria adapted to self-locomote while subject to a magnetic field, the magneto-aerotactic-responsive bacteria depositing the at least one of a treatment agent, an imaging agent and a diagnostic agent at hypoxic zones in a patient. The method includes adapting a total bolus escape time of a solution of the magneto-aerotactic responsive bacteria when injected into the patient in order to influence the targeting of a target zone with hypoxic zones in the patient, wherein decreasing the total bolus escape time favors a targeting, by the magneto-aerotactic-responsive bacteria, of one or more of the hypoxic zones at a farther distance from an injection site of the magneto-aerotactic-responsive bacteria while considering a decrease in velocity post-injection of said magneto-aerotactic-responsive bacteria when exposed to an environment of said patient.

In some embodiments, the total bolus escape time may be adapted by adjusting one or more of a concentration of the magneto-aerotactic-responsive bacteria in the solution; a volume of the bolus; a viscosity of the solution; and a surface of the bolus exposed to a north of the magnetic field when the magneto-aerotactic-responsive bacteria are north-seeking, or a surface of the bolus exposed to a south of the magnetic field when the magneto-aerotactic-responsive bacteria are south-seeking.

In some embodiments, the total bolus escape time may be adapted by dividing, during injection, the bolus into smaller boluses injected into the patient in order to lower a bolus-volume as the individual volumes of each of the smaller boluses is less than the volume of the bolus.

In some embodiments, increasing the total bolus escape time may be achieved by heating the solution in the injection device prior to introduction into the patient.

In some embodiments, increasing the total bolus escape time may be achieved by reducing the rate of volume of injection into the patient.

In some embodiments, the total bolus escape time may be adapted by adjusting the shape of the bolus.

In some embodiments, decreasing the total bolus escape time may be in order to enable access to a portion of a tumor that is inaccessible by the injection device, necessitating the travel of the magneto-aerotactic-responsive bacteria from the injection point to the portion of the tumor.

In some embodiments, the total bolus escape time may be adapted by influencing a shape of the bolus through a selection of an appropriate injector.

In some embodiments, the total bolus escape time may be decreased by increasing the surface to volume ratio of the injected bolus.

In some embodiments, an increase in total bolus escape time may be favoured when targeting of hypoxic zones closer to the injection site is sought.

Another broad aspect is a method of influencing a total bolus escape time of a solution of magneto-aerotactic-responsive bacteria attached to at least one of a treatment agent, an imaging agent and a diagnostic agent for injection into a patient, wherein the magneto-aerotactic-responsive bacteria is adapted to self-locomote while subject to a magnetic field, the magneto-aerotactic-responsive bacteria depositing the at least one of a treatment agent, an imaging agent and a diagnostic agent at a target zone with hypoxic zones in a patient. The method includes at least one of increasing the surface-volume ratio of the bolus, decreasing the concentration of the magneto-aerotactic-responsive bacteria in the solution, decreasing the viscosity of the solution, and increasing the surface of the bolus exposed to the north of the magnetic field for north-seeking magneto-aerotactic-responsive bacteria and to the south of the magnetic field for south-seeking magneto-aerotactic-responsive bacteria, that is to decrease the total bolus escape time of the magneto-aerotactic-responsive bacteria; or at least one of decreasing the surface-volume ratio of the bolus, increasing the concentration of the magneto-aerotactic-responsive bacteria in the solution, increasing the viscosity of the solution, and decreasing the surface of the bolus exposed to the north of the magnetic field for north-seeking magneto-aerotactic-responsive bacteria and to the south of the magnetic field for south-seeking magneto-aerotactic-responsive bacteria, that is to increase the total bolus escape time of the magneto-aerotactic-responsive bacteria, whereby a lower total bolus escape time of the magneto-aerotactic responsive bacteria is for targeting one or more of the hypoxic zones of the target zone in said patient that are further from said injection site.

In some embodiments, targeting of the one or more of the hypoxic zones in a tumor further from said injection site may be considered when the injection device cannot access a region next to said hypoxic zones, requiring a greater travel of the magneto-aerotactic-responsive bacteria from the injection site to the hypoxic zones, the method comprising at least one of increasing the surface-volume ratio of the bolus, decreasing the concentration of the magneto-aerotactic-responsive bacteria in the bolus, decreasing the viscosity of the bolus, and increasing the surface of the bolus exposed to the north of the magnetic field for north-seeking magneto-aerotactic-responsive bacteria and to the south of the magnetic field for south-seeking magneto-aerotactic-responsive bacteria to decrease the total bolus escape time of the magneto-aerotactic-responsive bacteria.

In some embodiments, the method may include at least one of increasing the surface-volume ratio of the bolus by dividing the bolus into smaller boluses.

In some embodiments, the magneto-aerotactic responsive bacteria may be attached to a treatment agent.

In some embodiments, the bolus may be prepared by further considering the velocity distribution of the magneto-aerotactic responsive bacteria in the bolus.

Another broad aspect is a container for supplying an amount of magneto-aerotactic-responsive bacteria into a subject for treatment, diagnosis and/or imagery using a magnetic field, the container comprising: an identifier adapted to be scanned for providing one or more properties of the magneto-aerotactic-responsive bacteria within the container; and the magneto-aerotactic-responsive bacteria located within the container.

In some embodiments, the one or more properties include one or more of: an activity decay of the magneto-aerotactic-responsive bacteria; a density of the magneto-aerotactic-responsive bacteria; a polarity or polarity ratio of the magneto-aerotactic-responsive bacteria; a reaction time of the magneto-aerotactic-responsive bacteria to a change in a magnetic field orientation; and a maximum speed of the magneto-aerotactic-responsive bacteria or speed distribution of the magneto-aerotactic-responsive bacteria.

In some embodiments, the identifier may be a barcode.

In some embodiments, the identifier may be a QR (quick response) code.

Another broad aspect is a method of characterizing one or more properties of magneto-aerotactic-responsive bacteria in a sample of magneto-aerotactic-responsive bacteria, comprising: generating a video of the magneto-aerotactic-responsive bacteria observed under a microscope while the magneto-aerotactic-responsive bacteria are subject to a magnetic field with a known magnetic field intensity and orientation; and analyzing a behavior of the magneto-aerotactic-responsive bacteria in the video to determine the one or more properties.

In some embodiments, the one or more properties include a speed distribution of the magneto-aerotactic-responsive bacteria, wherein the analyzing comprises calculating a distance travelled by magneto-aerotactic-responsive bacteria of the magneto-aerotactic-responsive bacteria over a period of time, and dividing the distance by the time.

In some embodiments, the one or more properties include an activity decay of the magneto-aerotactic-responsive bacteria, wherein the analyzing comprises, periodically at different time intervals, calculating a distance travelled by magneto-aerotactic-responsive bacteria of the magneto-aerotactic-responsive bacteria over a period of time, and dividing the distance by the time to measure a speed of the magneto-aerotactic-responsive bacteria at that given time, wherein the activity decay is determined from a decrease in the measured speed at the different time intervals.

In some embodiments, the one or more properties include a polarity of the magneto-aerotactic-responsive bacteria, wherein the analyzing comprises measuring a proportion of bacteria navigating in a direction corresponding to a magnetic north or magnetic south of the magnetic field.

In some embodiments, a viscosity of the solution of the bacteria may be determined by selecting a solvent composing the solution, by adjusting a concentration of a solute or salt present in a solution making up the solution, adjusting a concentration of magneto-aerotactic-responsive bacteria in solution, influencing the viscosity, etc.

### BRIEF DESCRIPTION OF THE DRAWINGS

The invention will be better understood by way of the following detailed description of embodiments of the invention with reference to the appended drawings, in which:

FIG. 1 is a drawing of an exemplary steerable magneto-aerotactic-responsive bacterium attached to an imaging agent, and an exemplary steerable magneto-aerotactic-responsive bacterium attached to a treatment agent;

FIG. 2 is a drawing of an exemplary steerable magneto-aerotactic-responsive bacterium attached to both an imaging agent and to a treatment agent;

FIG. 3 is a drawing of a map showcasing exemplary hypoxic zones through which the magneto-aerotactic-responsive bacteria will travel to from an injection site;

FIG. 4 is a drawing of another map showcasing exemplary hypoxic zones through which the magneto-aerotactic-responsive bacteria will travel to from an injection site;

FIG. 5 is an exemplary graph illustrating the velocity transpotherapeutic complex standard deviation of magneto-aerotactic-responsive bacteria joined to an agent (e.g. a therapeutic agent; a diagnostic agent; an imaging agent), where the velocity of the magneto-aerotactic-responsive bacteria is greater than a threshold velocity, indicative that at least most of the magneto-aerotactic-responsive bacteria will surpass one or more hypoxic zones when a magnetotaxis-favouring magnetic field intensity is applied, resulting in a more concentrated targeting of the magneto-aerotactic-responsive bacteria in the targeted hypoxic zones that can be further away from the injection site;

FIG. 6 is another exemplary graph illustrating the velocity transpotherapeutic complex standard deviation of magneto-aerotactic-responsive bacteria joined to an agent (e.g. a therapeutic agent; a diagnostic agent; an imaging agent), where only a portion of the magneto-aerotactic-responsive bacteria have a velocity greater than a threshold velocity, indicative of a greater spread or dilution of the magneto-aerotactic-responsive bacteria across a greater number of hypoxic zones when a magnetotaxis-favouring magnetic field intensity is applied;

FIG. 7 is a flowchart diagram of an exemplary method for modulating a magnetic field used for steering magneto-aerotactic-responsive bacteria for treatment, diagnosis and/or imaging of a subject; and

FIG. 8 is a block diagram of an exemplary system for targeted imaging, diagnosis and/or treatment of a subject with magneto-aerotactic-responsive bacteria;

FIG. 9A is a drawing of a representation of a large target volume at a target zone; and

FIG. 9B is a drawing of a representation of a small target volume at a target zone.

### DETAILED DESCRIPTION

The present disclosure describes methods of modulating a magnetic field used for steering magneto-aerotactic-responsive bacteria for treatment, diagnosis and/or imaging of a subject. The magneto-aerotactic-responsive bacteria are responsive to both a magnetic field (magnetotaxis) and an oxygen gradient (aerotaxis).

As a result, magnetic field intensity may be increased and/or decreased, depending on the location of the magneto-aerotactic-responsive bacteria, in order to change the movement pattern of the magneto-aerotactic-responsive bacteria, where a magnetic field intensity resulting in magnetotaxis promotes displacement from run-and-reverse motion (where changes of direction of movement of the bacteria with the direction of the magnetic field are reduced due to there being a run state that is greater than the relaxation time), and a magnetic field intensity resulting primarily in aerotaxis promotes changes of direction of movement of the bacteria from the magnetic field through run-and-tumble motion.

It will be understood that the magneto-aerotactic-responsive bacteria self-propel to locomote, steered by the torque applied by the magnetic field. Once it is determined or estimated that the magneto-aerotactic-responsive bacteria reach a target zone (e.g. of the tumor) with an oxygen gradient, the magnetic field intensity may be lowered. The lower magnetic field intensity allows the magneto-aerotactic-responsive bacteria to seek out hypoxic zones by following an oxygen gradient. The magneto-aerotactic-responsive bacteria follow the oxygen gradient, adopting more of a run-and-tumble pattern of movement, thereby allowing the magneto-aerotactic-responsive bacteria to migrate to the hypoxic zones. Once having located the hypoxic zones, the magneto-aerotactic-responsive bacteria remain at or near these hypoxic zones. Once the velocity of the magneto-aerotactic-responsive bacteria reaches 0 (e.g. perish or weaken in the subject), the magneto-aerotactic-responsive bacteria deposit the one or more agents (therapeutic; imaging; diagnostic) in the hypoxic zones. This results in targeted therapy, imagery and/or diagnosis, reducing the dosage of the agent required and the impact of the agent on the health of the subject due to the lower amount required.

Magneto-Aerotactic-Responsive Bacteria:

In the present disclosure, by magneto-aerotactic-responsive bacteria, it is meant a plurality of bacterium having flagella for self-propulsion and a chain of magnetosomes for direction, receptive to a magnetic field by orienting and migrating along geomagnetic field lines (i.e. magnetotactic). The magnetotactic quality of the magneto-aerotactic-responsive bacteria may be conferred by specific intracellular organelles called magnetosomes, which includenanometer-sized, membrane bound particles (crystals) of magnetic iron minerals and organized into chains via a dedicated cytoskeleton. The magnetic iron mineral particles may include iron oxide such as magnetite (Fe₃O₄) or iron sulfide such as greigite (Fe₃S₄) or pyrite (FeS₂), etc. The magneto-aerotactic-responsive bacteria also respond to an oxygen gradient (i.e. aerotactic). Examples of magneto-aerotactic-responsive bacteria are those of the species Magnetococcus marinus (e.g. strain MO-1, strain MSR-1, MC-1, BM-1, or genetically modified versions of species or strains, including those optimized in a laboratory, etc.) The magneto-aerotactic-responsive bacteria may be those that are naturally occurring, magneto-aerotactic-responsive bacteria that have been adapted through natural selection, or magneto-aerotactic-responsive bacteria that have been adapted through human intervention, such as in a laboratory setting, where certain properties of the bacteria may have been selected or optimized (e.g. the sensitivity of the bacteria to a magnetic field). Such human intervention may include, but is not limited to, genetic manipulation, selecting and reproducing bacteria that exhibit a certain property, tailoring the environment and/or growth media to optimize certain characteristics or obtain certain traits of the bacteria, etc. The magneto-aerotactic-responsive bacteria share the properties that they may be capable of self-propelling (in some cases, the magneto-aerotactic-responsive bacteria may be actuated by an external source that induces a magnetic torque to displace the magneto-aerotactic-responsive bacteria), the magneto-aerotactic-responsive bacteria may be magnetically guided and possess, and are adapted such that they can be guided, undergo motion and/or react to another external stimulus other than a magnetic field.

In an embodiment, the magneto-aerotactic-responsive bacteria according to the present disclosure are non-pathogenic, e.g., they do not proliferate or cause bacterial infection in the subject. The magneto-aerotactic-responsive bacteria may be from a non-pathogenic Magnetospirillum magneticum strain (e.g., AMB-1), a non-pathogenic Magnetotactic coccus strain, a non-pathogenic Magnetospirillum magnetotacticum strain, a non-pathogenic Magnetospirillum gryphiswaldense strain (e.g., MSR-1), a non-pathogenic Magnetospirillum bellicus strain, a non-pathogenic facultative anerobic magnetotactic spirillum strain, or non-pathogenic obligate anaerobe strain such as a non-pathogenic Desulfovibrio magneticus strain (e.g., RS-1). In an embodiment, the magnetotactic bacterium is a Magnetotactic coccus. It is to be understood that non-pathogenic bacteria may cause mild symptoms in the subject notably because of the presence of bacterial components that trigger some inflammation (e.g., LPS), but they are considered non-pathogenic if they do not proliferate or cause more severe symptoms in the subject.

The magneto-aerotactic-responsive bacteria have the ability to follow a decreasing oxygen gradient until they reach an oxygen level of less than about 2%. The normal oxygen levels in normal tissues (sometimes referred to as “physoxia”) vary between tissues, with an average of about 5-6% (ranging from about 7.5% to 4% depending on the tissue) (McKeown M A, Br J Radiol. 2014; 87(1035)). Thus, the magneto-aerotactic-responsive bacteria according to the present disclosure have the ability to migrate from an area of a tissue or organ that has normal oxygen levels (physoxia), e.g., from about 4% to about 7.5% or about 5-6%, to an area having reduced oxygen levels (hypoxia), for example an area of a tissue or organ in which oxygen levels are about 2% or less, about 1.75% or less, or about 1.5% or less. In an embodiment, the magneto-aerotactic-responsive bacteria according to the present disclosure have the ability to follow a decreasing oxygen gradient until they reach an oxygen level of about 0.3% to about 0.7%, about 0.4% to about 0.6%, or about 0.5%. This property is useful, for example, for reaching hypoxic areas in an organ, tissue or tumor. For example, most tumors typically have median oxygen levels of 2% or less, with some tumors having median oxygen levels of 1% or less (McKeown M A, Br J Radiol. 2014; 87(1035)). However, there is significant heterogeneity within individual tumors, and thus some areas of the tumors have oxygen levels below 1%, for example about 0.5%-1% or even lower. Such hypoxic areas of tumors are generally more resistant to cancer treatment, such as chemotherapy and/or radiotherapy.

In some examples, the magneto-aerotactic-responsive bacteria may survive only for a shortened period in the subject's body. The magneto-aerotactic-responsive bacteria may then die a short period after being administered to the subject. Therefore, when the magneto-aerotactic-responsive bacteria include a treatment agent, once the magneto-aerotactic-responsive bacteria die, the agent (e.g. treatment; diagnostic; imaging) may then remain at the site of interest.

Properties of the Magneto-Aerotactic Bacteria:

It will be understood that the magneto-aerotactic-responsive bacteria may be north-seeking (seeking the magnetic north), south-seeking (seeking the magnetic south) or a mixture of both. The magnetic field sequence(s) selected in accordance with the present disclosure may depend on if the magneto-aerotactic-responsive bacteria are north-seeking, south-seeking or a combination of both. A test may be performed on a sample or bolus of magneto-aerotactic-responsive bacteria prior to injection into the subject (e.g. by subjecting the magneto-aerotactic-responsive bacteria to a magnetic field with known properties, and monitor the response of the magneto-aerotactic-responsive bacteria to the magnetic field, using in some examples a microscope—a video of the behavior of the magneto-aerotactic-responsive bacteria may be generated using a camera and the microscope) to establish the traits of the magneto-aerotactic-responsive bacteria as being either south-seeking, north-seeking or a combination of both (a value of the ratio of south-seeking and north-seeking magneto-aerotactic-responsive bacteria may also be established).

In some instances, further traits of the magneto-aerotactic-responsive bacteria in a sample of magneto-aerotactic-responsive bacteria (e.g. to be administered to a subject) may be determined (e.g. using a microscope combined with a camera for generating a video of the magneto-aerotactic-responsive bacteria observed with the microscope). A speed of the magneto-aerotactic-responsive bacteria may be established by dividing a distance travelled (e.g. average distance travelled) by the magneto-aerotactic-responsive bacteria over time. A speed or velocity distribution may be established for a given swarm or sample of magneto-aerotactic-responsive bacteria. A response time of the magneto-aerotactic-responsive bacteria to a change in orientation of the magnetic field may also be determined by monitoring the reaction time of the magneto-aerotactic-responsive bacteria (i.e. time taken for the magneto-aerotactic-responsive bacteria to change direction of travel following the change in orientation of the magneto-aerotactic-responsive bacteria, in order for the magneto-aerotactic-responsive bacteria to be realigned with the new orientation of the magnetic field). The speed distribution (or speed) of the magneto-aerotactic-responsive bacteria, and/or the reaction time of the magneto-aerotactic-responsive bacteria in response to a change of orientation of the magnetic field may be used to determine a minimum magnetic field intensity for causing a displacement of the magneto-aerotactic-responsive bacteria (or a magnetic field intensity sufficiently low to enable the magneto-aerotactic-responsive bacteria to prioritize seeking out hypoxic regions in the subject).

A change in the orientation of the magnetic field may also provide information regarding the polarity of the magneto-aerotactic-responsive bacteria in the sample (i.e. north-seeking, south-seeking, or a mixture of both north-seeking and south-seeking).

A density of the magneto-aerotactic-responsive bacteria in a solution of a given sample may also be determined (e.g. from the video taken from the microscope, or from direct observation of the magneto-aerotactic-responsive bacteria using a microscope) by dividing a number of magneto-aerotactic-responsive bacteria by a known surface area or volume.

An activity decay of the magneto-aerotactic-responsive bacteria in a given sample may also be measured. A change in the speed of the magneto-aerotactic-responsive bacteria may be measured over time. The magneto-aerotactic-responsive bacteria may be observed using a camera (mounted to a microscope), a magnetic field source for applying a magnetic field to the sample containing the magneto-aerotactic-responsive bacteria, and with one or more sensors for characterizing the environment of the magneto-aerotactic-responsive bacteria (e.g. a pH monitor for measuring pH, a thermometer for measuring temperature, etc.). At given time intervals (e.g. every five minutes), a speed or speed distribution of the magneto-aerotactic-responsive bacteria in the sample may be calculated (i.e. by dividing a distance travelled by the magneto-aerotactic-responsive bacteria over time). The activity decay of the magneto-aerotactic-responsive bacteria may then be calculated by plotting a decrease in the measured speed of the magneto-aerotactic-responsive bacteria as a function of time (the speed calculated based from the distance over time measurements taken at the different time intervals). The activity decay of the magneto-aerotactic-responsive bacteria may be calculated at different environmental conditions (such as at 37 degrees C., the average temperature of a body of the subject).

The measured properties of the magneto-aerotactic-responsive bacteria may be stored in association with an identifier or code (e.g. a barcode or QR code), that is applied to a container for the sample of magneto-aerotactic-responsive bacteria. Scanning the identifier or code extracts the information defining the properties of the magneto-aerotactic-responsive bacteria in the given sample. These properties may be used to guide injection of the magneto-aerotactic-responsive bacteria into the subject, and/or apply an appropriate magnetic field or magnetic field sequences.

### Definitions

In the present disclosure, by “imaging”, it is meant a medical technique that allows for obtaining information on a subject. In some examples, the imaging allows for viewing of the anatomy of the subject. Such techniques include, but are not limited to, magnetic resonance imaging (MRI), computed tomography (e.g. CT scans, CAT scans), positron emission tomography, single photon-emission computed tomography, electron paramagnetic resonance, ultrasound, X-Rays, etc. In other examples, the imaging may be used to collect information on the subject, such as the presence of agents within or at a specific location within the subject. For instance, the imaging may be a radiation detector with a defined aperture (e.g. in some cases, a small aperture) capable of detecting radiation at a given part of the subject (e.g. such as near an organ).

In the present disclosure, by “bolus”, it is meant a dose of a solution that has been administered to a subject, thereby occupying a space in the subject at the injection point. It has a volume occupied in the subject at the injection point after or during injection. As such, the bolus has a given volume. In the present application, the bolus includes a concentration of magneto-aerotactic-responsive bacteria in solution that may be attached to an imaging agent, a diagnostic agent and/or an imaging agent. The shape of the bolus can be influenced by injection techniques, such as by moving the needle during injection, as the solution of magneto-aerotactic responsive bacteria is being introduced into the subject, or through the equipment used for the injection (e.g. choosing an injector with a specific head—a multi head injector, a single head injector, etc.)

In the present disclosure, by “diagnosing” or “diagnosis”, it is meant determining (i) the presence of a condition, disease or disorder, (ii) a risk of developing a condition, disease or disorder, or (iii) a state (aggravation, absence of change or improvement) of a condition, disease or disorder.

In the present disclosure, by “imaging agent”, it is meant an agent for providing more information about internal organs, cellular processes and tumors, as well as normal tissue. It may be a contrast agent that is capable of enhancing the contrast of structures or fluids that can be picked up using medical imaging. Such agents may include, but are not limited to, iodine, barium, iron oxide nanoparticle clusters, gadolinium and gadolinium derivative, magnetic nanoparticles such as iron platinum particles, manganese-based nanoparticles (e.g. manganese chelates), perflubron, fluorodeoxyglucose, protein-based imaging agents, microbubble imaging agents, etc. In some examples, the imaging agent may be present in a vehicle for containing the imaging agent (e.g. a vesicle). The imaging agent may be joined to a surface of the magneto-aerotactic-responsive bacteria, or incorporated therein. In some examples, the imaging agent may be an agent that emits radiation that can be picked up by a radiation detector (e.g. a radioisotope). In some examples, the imaging agent may be incorporated to the magneto-aerotactic-responsive bacteria, such as by growing, e.g., when the magneto-aerotactic-responsive bacteria are magnetotactic bacteria, the magneto-aerotactic-responsive bacteria in a growth medium with radioisotopes. In some examples, protein providing may also be integrated into the magneto-aerotactic-responsive bacteria. In some examples, the imaging agent emitting radiation may be jointed to a surface of the magneto-aerotactic-responsive bacteria.

In the present disclosure, by “subject”, it is meant mammal and non-mammals. Mammals mean any member of the mammalia class including, but not limited to, humans. Non-mammals include birds, reptiles, etc. The term “subject” should not bring on any limitations as to the sex or age.

In the present disclosure, by “target zone”, also referred to herein as “target zone”, it is meant a site in the subject towards which the magneto-aerotactic-responsive bacteria are to navigate. The target zone includes hypoxic zones towards which the magneto-aerotactic-responsive bacteria can move towards through aerotaxis, by following an oxygen gradient. The magneto-aerotactic-responsive bacteria may aggregate in the hypoxic zones of the target zone. The target zone may be a tumor, a portion thereof, an organ of the subject, where at least portions are the tumor includes hypoxic zones, etc. The target zone may include one or more hypoxic non-vascularized regions in the body of the subject resulting from or associated with, e.g., an ischemic stroke, pulmonary hypertension, ischemic cardiopathy, diabetic retinopathy, etc.

In the present disclosure, by “treating” or “treatment”, it is meant one or more of (i) preventing part or all of a condition, disease or disorder (temporarily or permanently), (ii) inhibiting or arresting part or all of a condition, disease or disorder (temporarily or permanently), and (iii) relieving part or all of a condition, disease or disorder (temporarily or permanently).

In the present disclosure, by “transpotherapy”, it is meant a therapy that is based on the directional-controlled or guided transport of therapeutic agents providing a therapeutic treatment modality capable of non-systemic delivery (such as using a more direct delivery routes between the injection (or entry) site and the physiological region to be treated) of therapeutics using transporters such as magneto-aerotactic-responsive bacteria at a specific physiological site with a specific volume. Unlike systemic delivery used in chemotherapy for instance, the main advantage of transpotherapy is to minimize systemic toxicity or systemic exposure while enhancing the therapeutic efficacy typically with much lower injected dosages but with a higher delivered dose to the site of treatment compared to chemotherapy. Transpotherapy can also transport therapeutics to the tumoral mass beyond the diffusion limit of standard chemotherapeutic agents. Here transpotherapy is used as a generic term and includes but is not limited to the non-systemic delivery of diagnostic (transpodiagnostic) and imaging (transpo-imaging) agents. Transpotherapy used for the delivery of radiosensitizers in radiotherapy for instance could be more specifically referred to as transporadiotherapy. Similarly, the use of transpotherapy in immunotherapy could be referred to as transpo-immunotherapy while transpochemotherapy would refer to the use of such transporters to achieve non-systemic chemotherapy. Combining treatment modalities using transpotherapy is also possible. One example among others is chemotherapy and immunotherapy that could be referred to as transpochemo-immunotherapy. A transpotherapeutic transporter loaded with both therapeutic and diagnostic agents could be referred to as a transpotheranostic complex. Many combinations are possible. As an example, transpochemo-imaging could be achieved with the same or separate injections of transpochemotherapeutic complexes combined with transpo-imaging (or transpocontrast) complexes; or using transpochemo-imaging complexes only within the same injection. The same type of nomenclature can be applied to other types of transpotherapeutic complexes or therapeutic treatment modalities, transpotherapy for the latter remaining the generic term.

In the present disclosure, by “velocity distribution”, also referred to herein as “velocity standard deviation”, it is meant the spread or extent of deviation in the velocities of a group of magneto-aerotactic-responsive bacteria found in a given sample or solution (e.g. for administration to a subject) under magnetotaxis. The velocity distribution is for the velocity pre-injection. The velocity distribution can be estimated or obtained using known imaging techniques (e.g. through microscopy) or by selection processes where cultures of magneto-aerotactic-responsive bacteria are produced and optionally combined as a function of their velocity (and samples or solutions can be further produced by selecting cultures demonstrating the sought-after velocity or velocity range).

The Magneto-Aerotactic-Responsive Bacteria:

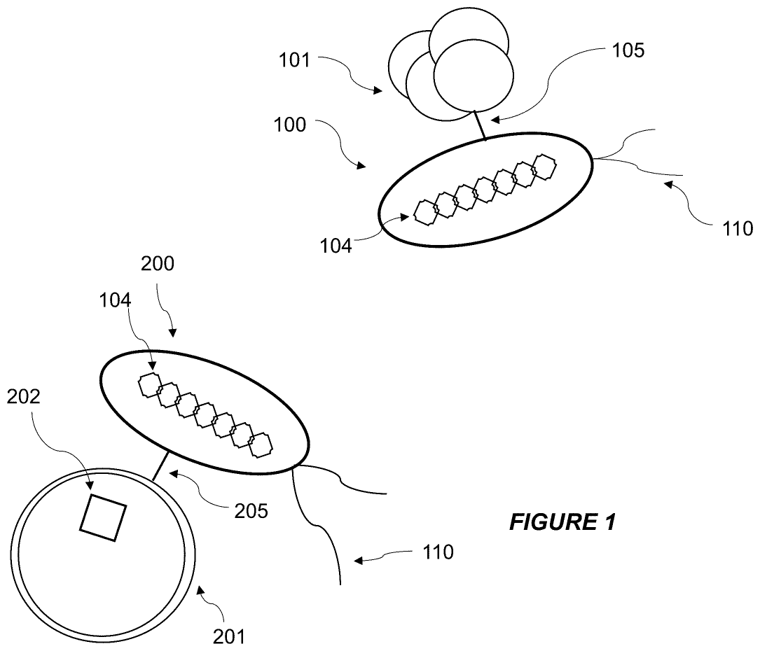

Reference is now made to FIG. 1, showing an exemplary simplified drawing of a magneto-aerotactic-responsive bacterium 100 and a magneto-aerotactic-responsive bacterium 200.

The magneto-aerotactic-responsive bacterium 100 and the magneto-aerotactic-responsive bacterium 200 have a chain of magnetosomes 104 that is sensitive to a magnetic field (such as an applied magnetic field, or the magnetic field of the Earth).

In some embodiments, the treatment agent 202 may be contained within a vehicle 201. The vehicle 201 may have a hydrophilic portion and a hydrophobic portion (e.g. a phospholipid bilayer). Depending on the hydrophobicity or the hydrophilicity of the treatment agent, the treatment agent may be present in either the hydrophilic or hydrophobic portion of the vehicle 201. In some examples, the treatment agent 202 may be contained in a biodegradable polymer vehicle. It will be understood that the treatment agent 202 may be contained in any vehicle for effectively containing of the treatment agent 202 for its delivery.

In some examples, the treatment agent 202 may be attached to the magneto-aerotactic-responsive bacterium 200 using, for instance, a ligand 205 (such as an antibody). The ligand 205 may be attached to the treatment agent 205, where the ligand may be adapted to bind with the exterior of the magneto-aerotactic-responsive bacterium 200. The ligand 205-treatment agent 202 may be placed in a solution with the magneto-aerotactic-responsive bacterium 200, where the ligand-treatment agent attaches to the magneto-aerotactic-responsive bacterium 200.

In some examples, the treatment agent 202 may be present in a vesicle 201, where the vesicle 201 may be joined to a ligand (e.g. antibody) adapted to join with the magneto-aerotactic-responsive bacterium 200. The vesicle 201 may then conjugate with the membrane of the magneto-aerotactic-responsive bacterium via the ligand 205.

In some examples, to enable the magneto-aerotactic-responsive bacterium 200 to stick to the vehicle 201 with the treatment agent 202, the magneto-aerotactic-responsive bacterium 200 may be forced to generate activation of lipopolysaccharides. For example, the magneto-aerotactic-responsive bacterium 200 may be mixed in a medium poor in concentration of nutrients prior to injecting a new concentration in a new medium containing a concentration of vehicles with treatment agents.

It will be understood that other mechanisms of fixing the treatment agent 202 to the magneto-aerotactic-responsive bacterium 200 may be used without departing from the present teachings. For instance, reference is made to Taherkhani et al., “Covalent Binding of Nanoliposomes to the Surface of Magnetotactic Bacteria for the Synthesis of Self-Propelled Therapeutic Agents”, ACS Nano. 2014 May 27; 8(5):5049-60, incorporated herein by reference.

Depending on the applications, other coupling means may be provided between the magneto-aerotactic-responsive bacterium and the treatment agents 202.

In some embodiments, the magneto-aerotactic-responsive bacterium does not have any vehicle 201, where the treatment agent 202 may be attached directly to the magneto-aerotactic-responsive bacterium 200, or may be incorporated within the magneto-aerotactic-responsive bacterium 200 (where the magneto-aerotactic-responsive bacterium may act as a vehicle for transporting the treatment agent).

In some examples, the treatment agent 202 and the imaging agent 101 may be the same (e.g. in the case of superparamagnetic iron-oxide nanoparticles).

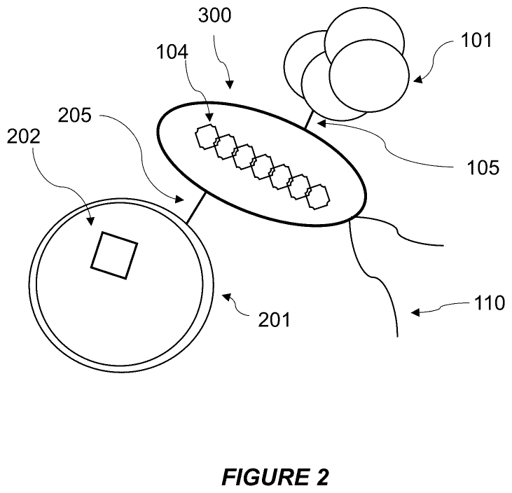

In some examples, the imaging agent 101 and the treatment agent 202 may be both fixed to the magneto-aerotactic-responsive bacterium (e.g. magneto-aerotactic-responsive bacterium 300 of FIG. 2).

In some examples, the imaging agent 101 may be attached to the magneto-aerotactic-responsive bacterium 100 using, for instance, a ligand 105 (such as an antibody). The ligand 105 may be attached to the imaging agent 101, where the ligand 105 may be adapted to bind with the exterior of the magneto-aerotactic-responsive bacterium 100. The ligand combined with the imaging agent may be placed in a solution with the magneto-aerotactic-responsive bacterium 100, where the ligand combined with the imaging agent may attach to the magneto-aerotactic-responsive bacterium 100.

It will be understood that other mechanisms of fixing the imaging agent 101 to the magneto-aerotactic-responsive bacterium 100 may be used without departing from the present teachings.

In some examples, the proportion, amount and/or concentration (or relative concentration) of the magneto-aerotactic-responsive bacterium and the magneto-aerotactic-responsive bacterium are known. When observing the amount of magneto-aerotactic-responsive bacterium that have reached and/or remain at a site of interest (e.g. at least one hypoxic zone of a tumor), and the amount (or proportion) of magneto-aerotactic-responsive bacteria that have dispersed away from the site of interest, based on the known proportion, quantity or concentration with respect to the magneto-aerotactic-responsive bacteria with treatment agent, it is possible to estimate that a similar proportion of magneto-aerotactic-responsive bacteria with treatment has remained at the site of interest, and the amount of magneto-aerotactic-responsive bacteria with treatment that has dispersed. For example, this information may provide an indication that is useful in assessing toxicity of a treatment agent (based on the amount of treatment agent that has dispersed through the body instead of being delivered to the site of interest). For example, this information may also be used to provide an indication as the dosage of the treatment agent required, where it is extrapolated that a proportion of the magneto-aerotactic-responsive bacteria with treatment agent, and therefore the carried treatment agent, does not make it or remain at the site of interest.

It will be understood that the solutions with the magneto-aerotactic-responsive bacteria may be prepared as is known in the art for preserving the magneto-aerotactic-responsive bacteria, and for further administration of the magneto-aerotactic-responsive bacteria to a subject.

In some examples, the magneto-aerotactic-responsive bacteria may be frozen prior to their joining to either or to both imaging agent and the treatment agent. The thawed magneto-aerotactic-responsive bacteria may be replicated and may be placed in a solution as described herein to join the magneto-aerotactic-responsive bacteria to the contrast agent, treatment agent or both.

Exemplary System for Targeting of an Aggregation Zone with Magneto-Aerotactic Bacteria:

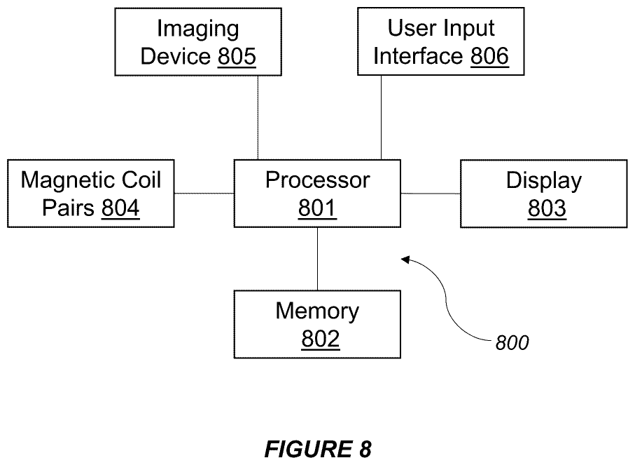

Reference is now made to FIG. 8, illustrating an exemplary system 800 for implementing a method for targeting of an aggregation zone in a subject with magneto-aerotactic-responsive bacteria by adjusting the magnetic field intensity of the magnetic field as a function of progress of the magneto-aerotactic-responsive bacteria in the body of the subject.

The system 800 includes a processor 801 and memory 802.

The system 800 is in communication with and may include one or more (e.g. three) pairs of magnetic sources 804 (e.g. magnetic coils) each aligned with respect to one of three axes (x, y and z). The system 800 is in communication with and may include an imaging device 805.

The system 800 may include a display 803. The system 800 may include a user input interface 806.

The processor 801 may be a general-purpose programmable processor. In this example, the processor 801 is shown as being unitary, but the processor 801 may also be multicore, or distributed (e.g. a multi-processor).

The computer readable memory 802 stores program instructions and data used by the processor 801. The memory 802 may be non-transitory. The computer readable memory 802, though shown as unitary for simplicity in the present example, may comprise multiple memory modules and/or cashing. In particular, it may comprise several layers of memory such as a hard drive, external drive (e.g. SD card storage) or the like and a faster and smaller RAM module. The RAM module may store data and/or program code currently being, recently being or soon to be processed by the processor 801 as well as cache data and/or program code from a hard drive. A hard drive may store program code and be accessed to retrieve such code for execution by the processor 801 and may be accessed by the processor 801 to store imaging information on the subject, values for velocities for the magneto-aerotactic-responsive bacterium, volumes of the injected bolus, program code for operating the magnetic sources 804, etc. The memory 802 may have a recycling architecture for storing, for instance, imaging information on the subject, etc., where older data files are deleted when the memory 802 is full or near being full, or after the older data files have been stored in memory 802 for a certain time.

The processor 801 and the memory 802 may be linked via BUS connections.

The user input interface 806 is for allowing a user to provide input to the system 800 to interact with the system 800, such as setting the magnetic field intensity generated by the magnetic sources 804, identifying on a display 803 the aggregation zone or the 3D convergence point, etc. The user input interface 806 may be a mouse, keyboard and/or controller and may be used to receive user input from the user.

It will be understood that other user input interfaces may be used in accordance with the present teachings, such as a touchscreen, a joystick, a microphone, one or more proximity sensor detecting movement of the user, etc.

In some examples, the system 800 may not have a user input interface 806, where the system 800 may receive user input provided on a remote computing device (e.g. remote computer, smartphone, tablet, laptop, etc.) via an input/output interface such as a transceiver (not shown).

The display 803 may provide a graphical user interface for controlling the system 800, may show to the user imaging information generated by the imaging device 805, parameters of the magnetic sources 804, etc. The display 803 may include a touchscreen function, thereby also acting as the user input interface 806.

The magnetic sources 804 may include one or more pairs of magnetic coils.

Each of the pairs of magnetic coils may be aligned along one axis. For instance, the magnetic sources 804 may feature three pairs of magnetic coils, where each of three pairs of magnetic sources 804 is aligned with respect to one of the three axes (x, y and z). The magnetic sources 804 may generate the 3D convergence point (as well as the aggregation zone) as described herein or in U.S. Pat. No. 9,905,347.

The imaging device 805 generates imaging information on the subject, e.g. for defining the aggregation zone, the 3D convergence point, providing visual information on the body of the subject (e.g. tumor), providing visual information on the hypoxic zones of the tumor, monitoring the movement of the magneto-aerotactic-responsive bacteria once injected in the subject, etc. The imaging device 805 permits the implementation of an imaging as described herein. For instance, the imaging device 805 may be a PET (positron emission tomography) machine, an MRI (magnetic resonance imaging) machine, a CT (computed tomography) machine, etc.

Exemplary Method of Targeting with Magneto-Aerotactic-Responsive Bacteria:

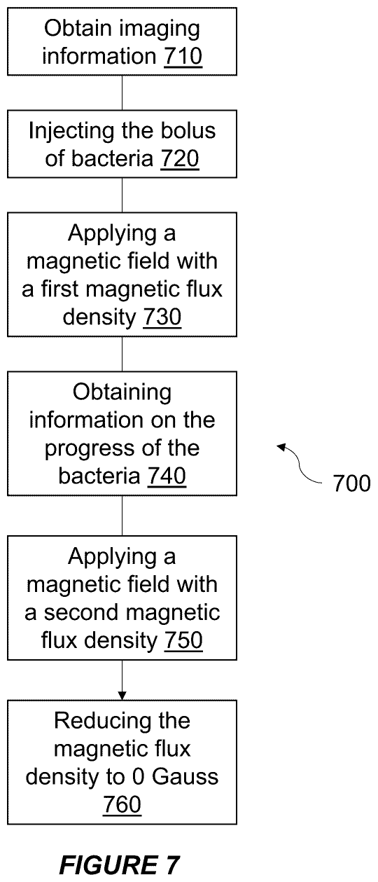

Reference is now made to FIG. 7, illustrating an exemplary method 700 of targeting an aggregation zone in a subject with magneto-aerotactic-responsive bacteria by modulating the magnetic field intensity of the magnetic field.

For illustrative purposes, with reference to the exemplary system 800, the method 700 may be implemented by the processor 801, executing program code stored in memory 802, the processor 801 carrying out the instructions of the program code stored in memory 802. The processor 801 may regulate power/current being provided to the magnetic sources 804 in order to regulate the magnetic field intensity of the magnetic field generated by the magnetic sources 804.

The processor 801 may also control the imaging device 805 in order to obtain imaging information. In some embodiments (e.g. when the imaging device 805 is not part of the system 800), the imaging information may also be transmitted to the system 800, and to the processor 801 through an input/output interface (I/O) (e.g. a transceiver).

Imaging information is obtained on a subject at step 710. Imaging information may be obtained through imaging as defined herein. The imaging information may provide information on a target zone, tissue or organs of the subject (e.g. a tumor, the anatomy of the subject or a portion thereof, the hypoxic zones of the tumor, etc.)

In some embodiments, imaging information can be enhanced through the use of magneto-aerotactic-responsive bacteria combined with an imaging agent. The imaging agent can be picked up by the imaging, thereby enhancing the information gathered through use of the imaging.

One or more boluses of the magneto-aerotactic-responsive bacteria are injected at one or more sites on the subject at step 720. In some embodiments, the magneto-aerotactic-responsive bacteria may be injected into a peripheral region of a tumor. In some embodiments, the injection may be systemic. The bolus has a given concentration of magneto-aerotactic-responsive bacteria and a given volume as the solution of magneto-aerotactic-responsive bacteria is injected in the patient. The magneto-aerotactic-responsive bacteria may be tethered to one or more of a therapeutic agent (e.g. an anti-cancer agent), a diagnostic and/or an imaging agent.

Applying a first mode, a magnetic field is applied having a magnetic field intensity sufficient for applying a directional torque on the magneto-aerotactic-responsive bacteria at step 730, where motion of the bacteria is dominated by magnetotaxis and run-and-reverse motion (less change of direction of movement of the bacteria from run-and-tumble as the increased run state and lower relaxation time permits realignment of the direction of movement of the bacteria with the direction of the magnetic field). This first mode is to achieve displacement of the bacteria from their current location (e.g. injection site) to a new location, e.g. target zone. In some embodiments, this first magnetic field intensity may be of at least 15 Gauss. However, a skilled person in the art will readily understand that the value of the first magnetic field intensity may vary depending on the properties of the magneto-aerotactic-responsive bacteria, such as their sensitivity to the magnetic field. The magnetic field intensity may be calculated from and may depend on the volume of the bolus of the injection, the distance travelled by the magneto-aerotactic-responsive bacteria (e.g. can be calculated by the known velocity of the magneto-aerotactic-responsive bacteria and the time lapsed from injection), the time lapsed from injection (as the speed and responsiveness of the bacteria decrease as a function of the time spent in the human body), surface area of the bolus and/or the concentration of magneto-aerotactic-responsive bacteria in the bolus.

The magnetic field may steer the magneto-aerotactic-responsive bacteria in one to three dimensions, by e.g. generating a 3D convergence point as described herein.

Optionally, information on the progress of the magneto-aerotactic-responsive bacteria as they locomote in the subject may be generated (e.g. through monitoring of the movement of the magneto-aerotactic-responsive bacteria; estimating the position of the magneto-aerotactic-responsive bacteria) at step 740. For instance, the monitoring may be performed by having the magneto-aerotactic-responsive bacteria combined with an imaging agent that is perceptible through imaging. In some examples, the monitoring may be performed by calculating the time lapsed from the injection time of the magneto-aerotactic-responsive bacteria into the subject. Distance can be estimated from the velocity distribution of the bacteria pre-injection, the regression in velocity of the bacteria post-injection, the time lapsed since injection. In some examples, the distance value may also be calculated from the volume and surface area of the injection bolus.

In a second mode, a magnetic field with a second magnetic field intensity that is lower than the first magnetic field intensity is applied when it is observed or estimated that the magneto-aerotactic-responsive bacteria are approaching a target zone, the target zone having or being near oxygen gradients resulting from the hypoxic zones at the target zone at step 750. This lowering of the magnetic field intensity causes a shift in the kind of motion of the magneto-aerotactic-responsive bacteria. The magneto-aerotactic-responsive bacteria adopt more of a run-and-tumble motion than with the higher first magnetic field intensity is applied. This change in motion type to run-and-tumble permits the magneto-aerotactic-responsive bacteria, through aerotaxis, to seek out the hypoxic zones of the tumor. In some examples, this second magnetic field intensity may be less than 15 Gauss but equal to or greater than 5 Gauss. However, a skilled person in the art will readily understand that the value of the second magnetic field intensity may vary depending on the properties of the magneto-aerotactic-responsive bacteria, such as their sensitivity to the magnetic field. The magnetic field intensity may be calculated from and may depend on the volume of the bolus of the injection, viscosity, shape of bolus, anisotropic orientation, the distance travelled by the magneto-aerotactic-responsive bacteria (e.g. can be calculated by the known velocity of the magneto-aerotactic-responsive bacteria and the time lapsed from injection), the time lapsed from injection (as the speed and responsiveness of the bacteria may decrease as a function of the time spent in the human body), the concentration of magneto-aerotactic-responsive bacteria in the bolus.

In some embodiments, in a third mode, once the magneto-aerotactic-responsive bacteria have reached oxygen gradients of the hypoxic zones of the target zone, the magnetic field intensity of the magnetic field may be reduced to less than 5 Gauss and down to 0 Gauss at step 760. This reduction of the magnetic field intensity is to further maintain the magneto-aerotactic-responsive bacteria in the hypoxic zones of the target zone, the magneto-aerotactic-responsive bacteria further change direction, unaligned with the direction of the magnetic field, as a result of run-and-tumble motion than at step 750. As the magneto-aerotactic-responsive bacteria may be tethered to one or more therapeutic agent(s), diagnostic agent(s) and/or imaging agent(s), once the post-injection velocity of the magneto-aerotactic-responsive bacteria is reduced to 0 (e.g. as they perish or are weakened) in the hypoxic zones (e.g. several minutes after their introduction into the subject), the therapeutic agent(s), diagnostic agent(s) and/or imaging agent(s) are deposited in the hypoxic zones of the subject. This results in targeted administration of compounds to the subject. Moreover, this method enables specific targeting of hypoxic zones of tumors, proven difficult through traditional administration of compounds to the subject as a result of the vasculature and cellular structure of the tumor.

The method leverages the change in responsiveness and integrity of the magneto-aerotactic-responsive bacteria once they are administered to the subject. As the magneto-aerotactic-responsive bacteria require colder temperatures and certain environmental conditions to survive that are not offered by the human body, the introduction of the magneto-aerotactic-responsive bacteria in the human body results in shorter lifespan therein. Moreover, the longer the time the magneto-aerotactic-responsive bacteria spend in the human body, the more sluggish they become as they eventually die off. As such, precision of targeting while leveraging the time when the magneto-aerotactic-responsive bacteria travel at their higher speed (after injection) is advantageous. Therefore, the higher magnetic field intensity, up to maximum value (to avoid repolarization issues) applied after injection favours the magnetotactic response of the magneto-aerotactic-responsive bacteria, allowing the magneto-aerotactic-responsive bacteria to travel the greater distance required to reach the oxygen gradients next to the hypoxic zones of the target zone as the magneto-aerotactic-responsive bacteria show optimal activity. Once the magneto-aerotactic-responsive bacteria approach the oxygen gradients produced by the tumor, the magneto-aerotactic-responsive bacteria are slower due to the hostile environment of the subject's body. However, this sluggishness is acceptable as the magneto-aerotactic-responsive bacteria may travel lesser distances, as the magneto-aerotactic-responsive bacteria seek out the hypoxic zones of the tumors.

Exemplary Method of Controlling Dispersion of Magneto-Aerotactic Bacteria when Targeting:

When performing the targeting as described, e.g., with respect to exemplary method 800, it may be preferable to reach a greater number of hypoxic zones per injection, while reducing the concentration of bacteria per hypoxic zone, at the target zone, or achieve concentrated targeting of a few hypoxic zones with a greater concentration of bacteria. There may be an advantage in adapting the targeting volume at the target zone depending on the desired result, where a more dispersed targeting with the bacteria would result in a greater targeting volume, while a less dispersed (and more concentrated) targeting with bacteria would result in a lesser targeting volume at the target zone.

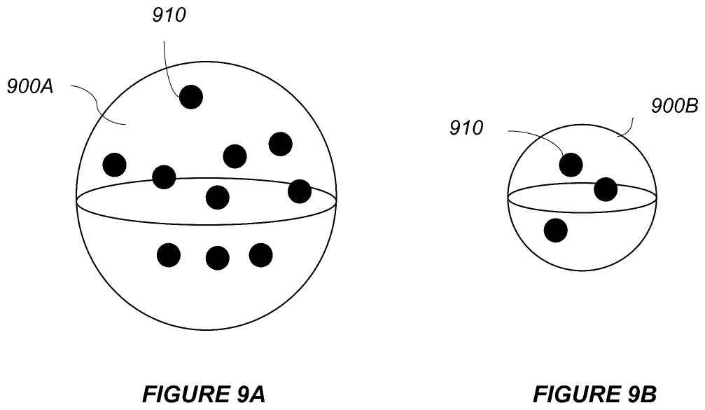

In order to increase or decrease the targeting volume at the target zone reached by the injected bacteria, this may be achieved by preparing or choosing a sample of bacteria to administer based on the sample's distribution velocity. Reference is made to FIG. 9A, illustrating an exemplary larger target volume 900A with multiple hypoxic zones 910 at a target zone. FIG. 9B illustrates a small target volume 900B with less hypoxic zone at a target zone. It will be understood that the exemplary target volumes 900A and 900B are simplified for purposes of illustration, as well as the hypoxic zones, and may adopt a variety of shapes depending on the subject's anatomy and the organ, tissue or tumor that is targeted. Target volumes 900A and 900B are to illustrate a difference of magnitude in size between target volumes. For target volume 900A, a sample of bacteria with a greater velocity distribution may be selected. For target volume 900B, a sample of bacteria with a narrower velocity distribution may be selected.

A greater velocity distribution would result in a greater targeting volume or dispersion. A lesser velocity distribution would result in a lower targeting volume or dispersion.

Assessing this velocity distribution (e.g. standard deviation in the velocities of the bacteria of the sample) may allow a user to select a sample based on the desired targeting volume at the target zone.

Furthermore, the velocity pre-injection of the bacteria in the sample may also be compared to a threshold velocity value for overcoming one or more hypoxic zones near the injection site. Bacteria exhibiting a velocity greater than the threshold may travel past those hypoxic zones, where bacteria with a velocity lower than the threshold may remain aggregated in those hypoxic zones. As such, the velocities themselves of the bacteria pre-injection (in addition to their velocity distribution) may provide information as to the travel pattern of the bacteria post-injection.

The velocity distribution of a sample can be assessed (and, in some instances, determined if not provided by the manufacturer of the bacteria sample) prior to injection into the patient. If the velocity distribution of the sample does not correspond to what is sought for achieving the desired targeting volume at the target sit, the sample may be discarded or exchanged with another exhibiting a more desirable velocity distribution.

Moreover, a sample may be produced as a function of the desired dispersion or targeting volume at the target zone. For instance, a plurality of bacterial cultures may be grown, where each of the bacterial cultures may be associated with a given velocity or velocity range (e.g. “slow”; “medium”; “fast”). If a small velocity distribution is desired, a sample can be prepared from one culture demonstrating relatively similar velocities. However, if a large velocity distribution is sought, the sample can be prepared from multiple cultures, where each culture demonstrates a different velocity level, thereby including slower and faster bacteria (increasing the velocity distribution of the overall prepared sample). It will be understood that velocity ranges can also be selected from a same culture.

In other embodiments, a user may focus on the velocity regression post-injection to select a sample. For instance, some bacteria may be more resistant to the environment in the subject and therefore may take longer to lose their velocity. In other instances, the bacteria may instead be less resistant and therefore lose velocity rapidly post-injection. With a known profile of the expected velocity regression post-injection, a user may choose a sample with a greater range of changes in velocity post-injection when the target volume is greater (even if the velocity distribution is relatively narrow), and choose a sample with a smaller range of changed in velocity post-injection when the target volume is lesser. Once the bacteria have been administered to the subject, with a sample showing a greater range of changes in velocity post-injection, the spread in change in velocity post-injection will result in a spread in the exhibited velocities over time, thereby increasing the distance covered the bacteria when the magnetic field intensity is modulated to favour magnetotaxis and motion through run-and-reverse of the bacteria.

Exemplary Method of Improving the Targeting of Magneto-Aerotactic Responsive Bacteria Through Adjustment of the Total Bolus Escape Time:

Total Bolus Escape Time refers to the total dissolution time of the injected bolus of magneto-aerotactic responsive bacteria, i.e. the total time needed for all motile magneto-aerotactic responsive bacteria to escape the injected bolus.

The total bolus escape time can be a factor used to improve a targeting of a region in a patient using the magneto-aerotactic responsive bacteria. This improvement in targeting is achieved when the total bolus escape time is considered along with the velocity post-injection (or the velocity after the bacteria has been placed in an environment that negatively impacts its mobility, such as a human body) of the magneto-aerotactic responsive bacteria in the patient. More particularly, the longer the time taken for the magneto-aerotactic responsive bacteria to leave the bolus (at the injection site), the longer the magneto-aerotactic responsive bacteria spend in an environment that reduces the mobility (and velocity) of the bacteria. As such, the longer it takes for the magneto-aerotactic responsive bacteria to escape, the slower the magneto-aerotactic responsive bacteria will be, as their velocity will have at least partially regressed due to this delay.

The injection may occur inside the target zone (e.g. intratumoral) or around the target zone (e.g. peritumoral).

As a result, a shorter total bolus escape time provides for a larger targeted volume at a greater distance from the injection site. This is because the injected magneto-aerotactic responsive bacteria will have a greater velocity when injected into the patient and escape the bolus, and will be able to therefore travel a greater distance post-injection due to this higher velocity. Maintaining a short total bolus escape time (whereby the velocity post-injection of the bacteria is high) may be useful for targeting areas in the patient that are not as easily accessible by the needle or the injection device. For instance, if an injection device cannot access one side of a tumor or an organ, the injection device can instead administer the magneto-aerotactic responsive bacteria at a location of the tumor or organ that is accessible by the injection device (but at a distance from the target location), and then the magnetic field can direct the magneto-aerotactic responsive bacteria to the target location, the magneto-aerotactic responsive bacteria benefitting from a velocity post-injection sufficient for self-locomotion to the target location.

In contrast, a longer total bolus escape time, resulting in at least a partial decrease of the velocity post-injection of the magneto-aerotactic responsive bacteria, can be privileged when the target zone is next to the injection point. In these embodiments, it is preferred for the magneto-aerotactic responsive bacteria to remain close to the injection point post-injection. As a result, a reduced velocity-post injection may be preferred, where the magneto-aerotactic responsive bacteria will have limited displacement post-injection. The magneto-aerotactic responsive bacteria will remain near the injection point, near the bolus, and near the target zone.

In some examples, reducing the speed of the magneto-aerotactic responsive bacteria prior to the injection may increase the total bolus escape time (e.g. when in the injection device), such as by, heating the solution above room temperature (but preferably below body temperature) prior to injecting into the subject can increase the total bolus escape time exposing the solution to ultraviolet rays, etc.

It will be understood that adjusting or taking into account the total bolus escape time when targeting a location in a patient using the magneto-aerotactic responsive bacteria, may be used alone with or in combination with any of the other parameters and/or techniques described herein, such as modulating the magnetic field intensity, selecting a sample of magneto-aerotactic responsive bacteria with a specific velocity distribution profile, etc.

Impacting the total bolus escape time can be achieved when preparing the solution of magneto-aerotactic responsive bacteria to be administered to the patient (either by the medical professional who will administer the bolus, or a manufacturer of the solution of magneto-aerotactic responsive bacteria), where properties of the bolus can be selected to impact the total bolus escape time.

For instance, such properties include, but are not limited to, the concentration of the bolus, the viscosity of the bolus (can be related to its concentration).

Other properties that may influence the total bolus escape time may be adjusted during injection (e.g. by the medical specialist administering the solution of magneto-aerotactic responsive bacteria to the patient), namely the surface area to volume ratio of the bolus, the surface area of the bolus that is exposed to the north of the magnetic field for north-seeking magneto-aerotactic-responsive bacteria and to the south of the magnetic field for south-seeking magneto-aerotactic-responsive bacteria, the volume of the bolus, etc. These properties may be influenced by the injection speed (volume injected as a function of time), if the syringe is pulled away from the initial injection point as the solution of magneto-aerotactic response bacteria continues to be introduced into the body, etc.

Therefore, the total bolus escape time may also be modified by delaying administration of the bolus into the patient (e.g. where the solution including the magneto-aerotactic responsive bacteria is retained in the injection device for a specific period of time). The choice of the type of injector used for the injection may also influence the properties of the bolus (e.g. the shape of the bolus, the surface area of the bolus, etc.) Exemplary injectors include, but are not limited to, multi-hole injectors, single hold injectors, multi side hole injectors, etc.

The concentration of the magneto-aerotactic responsive bacteria in the solution for injection can impact the viscosity of the solution, and thereby also the total bolus escape time. A greater concentration results in a greater viscosity, and vice-versa. A sample with a greater concentration will result in an increased total bolus escape time due to the increased viscosity. A sample with a lower concentration will result in a lower total bolus escape time due to the lowered viscosity.

The viscosity of the bacterial solution (and therefore also the total bolus escape time) may also be impacted by one or more other ingredients added to the bolus (e.g. the properties of the excipient of the bolus).

The surface area to volume ratio of the bolus can also impact the total escape time of the bolus, where a greater surface area to volume ratio results in a decreased total bolus escape time. In contrast, a smaller surface area to volume ratio can result in an increased total bolus escape time. The surface area to volume ratio can be adjusted by changing the shape of the bolus (e.g. spherical vs. cylindrical), or by dividing the volume of a bolus into multiple smaller boluses, where each of the smaller boluses is injected independently into the patient.

In some embodiments, the proportion of the surface area of the bolus that is facing the north of the magnetic field for north-seeking magneto-aerotactic-responsive bacteria and to the south of the magnetic field for south-seeking magneto-aerotactic-responsive bacteria may also impact the total bolus escape time. A greater ratio of the surface area facing the north of the magnetic field for north-seeking magneto-aerotactic-responsive bacteria and to the south of the magnetic field for south-seeking magneto-aerotactic-responsive bacteria would promote evacuation of the magneto-aerotactic responsive bacteria and therefore decrease the total bolus escape time. In contrast, a smaller ratio of the surface area facing the north of the magnetic field for north-seeking magneto-aerotactic-responsive bacteria and to the south of the magnetic field for south-seeking magneto-aerotactic-responsive bacteria would impede evacuation and increase the total bolus escape time.

It will be understood that magneto-aerotactic-responsive bacteria may be polar or axial, where polar magneto-aerotactic-responsive bacteria may be north-seeking or south-seeking, where axial magneto-aerotactic-responsive bacteria may be both south-seeking and north-seeking, where the axial magneto-aerotactic-responsive bacteria may move along a north-south axis, and may change direction along the axis when confronted, e.g., with obstacles.

A greater volume can also result in a greater total bolus escape time when the injection device can only dispense a certain volume of the solution as a function of time. Decreasing the volume can therefore decrease the total bolus escape time. However, if the decreasing of the volume results in an increased concentration, and therefore an increased viscosity, this may also increase the total bolus escape time.

The total bolus escape time can also be adjusted by reducing the injection rate (e.g. volume injected into the patient as a function of time) into the patient, where a decreased injection rate would increase the total bolus escape time. In contrast, an increased injection rate would decrease the total bolus escape time.

It will be understood that properties of the bolus that may impact the total bolus escape time described herein are but exemplary and that other properties of the bolus may also impact the total bolus escape time.

Certain of the properties of the bolus that can impact the total bolus escape time may be adjusted by a manufacturer who is preparing the solution of magneto-aerotactic responsive bacteria for injection into a patient (e.g. in vials or injection devices). Such properties include, but are not limited to, the concentration of the bolus, the viscosity of the bolus, etc.

Some properties of the bolus may be adjusted by the medical specialist who is administering the bolus. Such properties include, but are not limited to, the surface-area to volume ratio of the bolus, the volume of the injected bolus, the shape of the injected bolus, etc. These properties of the bolus may be influenced by the injection techniques used by the medical specialist, or the tools used (e.g. type of injector) used by the medical specialist performing the injection. For instance, a greater bolus volume may be achieved by the medical practitioner gradually moving the point of the injector during the course of the injection. As a result, the magneto-aerotactic responsive bacteria are not injected at the same point in the subject, therefore increasing the volume of the injected magneto-aerotactic responsive bacteria in the patient. Depending on the orientation, displacement, rotation, etc. of the injector during the injection, these movements may also influence the shape of the bolus, and therefore affect the total bolus escape time. In some embodiments, the nature (e.g. density, shape, etc.) of the tissue at the injection point may also influence the shape of the bolus, where the injected solution may pocket in a denser envelope of tissue. As such, the environment tissue may also be used to adjust the shape of the bolus, and therefore also the total bolus escape time.

It will also be understood that the medical specialist may also delay injection of the bolus into the patient, thereby increasing the total bolus escape time.

Exemplary Study:

The following exemplary study is provided to enable the skilled person to better understand the present disclosure. As it is but illustrative and representative examples, it should not limit the scope of the present disclosure, only added for illustrative and representative purposes. It will be understood that other exemplary studies may be used to further illustrate and represent the present disclosure without departing from the present teachings.

Tumoral regions with low oxygen condition known as Hypoxic Zones (HZ) render tumor cells resistant to various treatment modalities such as chemo- and radiotherapy while resulting in a more malignant and invasive phenotype leading to a negative prospect in subject prognosis. For instance, the low oxygen level in hypoxic zones renders radiotherapy inefficient while chemotherapeutic agents cannot reach adequately the deeply located hypoxic zones due mainly to diffusion limit. Therefore, targeting hypoxic zones with special propelling vectors capable of transporting therapeutics or other cargo to hypoxic zones and referred to here as Transpotherapeutic Transporters (TT) and Transpotherapeutic Complexes (TC) when considering the transpotherapeutic transporters with the cargos, would enhance substantially the therapeutic outcomes.

Specific magneto-aerotactic-responsive bacteria, as described herein, can be well suited to act as transpotherapeutic transporters for targeting and transporting payloads to hypoxic zones. These magneto-aerotactic-responsive bacteria cells are typically microaerophilic or the like that seek oxygen levels close or equivalent to the ones found in tumor hypoxic zones. Since tumor hypoxia generally occurs at oxygen level <1%, and often <0.1% oxygen for a limited period (7.5 to <0.75 mmHg) (Br J Radiol. March 2014; 87(1035): 20130676), the Target Oxygen Level (TOL) defined here as the oxygen level being targeted by the transpotherapeutic transporters or transpotherapeutic complexes while operating in the Aerotactic Targeting Mode (ATM), i.e., the directional displacement of the transpotherapeutic complexes is influenced by oxygen gradients without the influence of another source such as a directional magnetic field capable of influencing its directional swimming behavior, may be appropriate or sufficiently close to hypoxic zones to yield a suitable target therapeutic effect beyond what is possible with other conventional treatments such as radiotherapy and chemotherapy, to mention but only two main treatment modalities.

Magneto-aerotactic transpotherapeutic transporters or transpotherapeutic complexes are most often guided towards regions with hypoxic zones using various Magnetic Field Configurations (MFC). One or a sequence of magnetic field configurations known as a Magnetic Field Configuration Sequence (MFCS) forms a Transpotherapeutic Targeting Sequence Event (TTSE) (or simply referred to as event) using one of three Magnetotactic Targeting Modes (MTM) (Direct Magnetotactic Targeting (DMT), Spatially-limited Magnetotactic Targeting (SMT), and Aggregation Zone Targeting (AZT)) or their derivatives leading to a specific directional Displacement Path (DP) of the transpotherapeutic complexes while a sequence of all the required transpotherapeutic targeting sequence events results in TTS (the targeting sequence). Within the magnetic field configurations, the transpotherapeutic complex is submitted to a Directional Vector (DV) that will cause the transpotherapeutic complex to follow a specific displacement path. Each directional vector at a given time is constituted of two components being the magnitude and the direction of magnetic field configurations applied to the transpotherapeutic complex. For magnetic field configurations, the magnitude is the magnetic field intensity. With regard to hypoxic zones and the related influence on the motion behavior of the transpotherapeutic complexes with regard to the desired directional displacements of the transpotherapeutic complex, magneto-aerotactic targeting methods (MATM) exploiting the magnetic field intensity in the directional vector are used to adjust the magnitude of the magnetic field configurations appropriately to target hypoxic zones by considering some specific main parameters that are listed in the following sections.

Main Parameters

Magnetic Field Intensity Range (FIR)

These magneto-aerotactic-responsive bacteria transpotherapeutic transporters that are suitable for targeting hypoxic zones are typically motile magneto-aerotactic bacteria, meaning here that they can travel and operate in a magnetotactic mode only, an aerotactic mode only, or both simultaneously. The selection of the appropriate displacement or targeting mode of the magneto-aerotactic-responsive bacteria transpotherapeutic transporters is done by exposing the transpotherapeutic transporters or transpotherapeutic complexes to a specific range of directional magnetic flux densities. Magnetic field intensity Ranges in order of increasing densities that are referred to here as Aerotactic Magnetic field intensity Range, Magneto-Aerotactic Magnetic field intensity Range and Magnetotactic Magnetic field intensity Range are hereby defined for aerotactic, magneto-aerotactic, and magnetotactic displacement or targeting mode respectively. Although these ranges can vary among transpotherapeutic complexes, specific physiological environments, and other factors, a typical magneto-aerotactic magnetic field intensity range may range between approximately 5 and 15 Gauss and be bordered at the lower and upper ends by AEROTACTIC FIELD INTENSITY RANGE (with a lower bound at 0 Gauss) and magnetotactic magnetic field intensity range (with an upper bound being limited by potential repolarization (RPL) of transpotherapeutic transporters that would reverse the intended direction of displacement of the transpotherapeutic transporters at higher magnetic field intensity), respectively.

The magneto-aerotactic-responsive bacteria acting as transpotherapeutic transporters perform aerotaxis using a combination of run-and-tumble and run-and-reverse motions. The chain of magnetosomes containing magnetic particles in the magneto-aerotactic-responsive bacterium cell has a magnetic moment in the order of 10⁻¹⁵ A m², allowing a relatively weak directional magnetic field to induce a magnetic torque which tends to align the direction of motion of the magneto-aerotactic-responsive bacterium cell along the direction of the magnetic field. But tumbling perturbs this alignment and as such, the run-and-tumble motion is characterized by perturbations of this alignment due to tumbles, followed by relaxation to the alignment with the directional magnetic field during the run state. If the run state is shorter than the relaxation time, the magnetic field does not have sufficient time to realign the magneto-aerotactic-responsive bacterium cell before the next tumble causing the direction of motion of the magneto-aerotactic-responsive bacterium cell to fluctuate from the direction of the magnetic field. The viscosity of the media and the geometrical properties of the transpotherapeutic complexes are just two factors among others that can influence the relaxation time. For instance, since the rotational friction coefficient is directly proportional to the viscosity (n) of the media, more induced torque is required to turn the cell, and thus the distribution of tumble angles is expected to be skewed towards small angles. Increasing the intensity of the directional magnetic field increases the magnetic torque induced on the chain of magnetosomes and therefore reduces the relaxation time, yielding a decrease of the fluctuation amplitudes from the direction of the magnetic field.

Within magneto-aerotactic magnetic field intensity range, the directional displacement of the transpotherapeutic complex is still influenced by the directional magnetic field but experiences a longer relaxation time especially at the lower magnetic field intensity levels, resulting in an increase of fluctuations of the swimming directions from the direction of the magnetic field. This increase in fluctuations contributes to maintaining the transpotherapeutic complex in the tumor hypoxic zones when transiting through the hypoxic zones at least temporarily in the magneto-aerotactic magnetic field intensity range and up and beyond the total transpotherapeutic complex Motility Time (MT) when the magnetic field intensity is lowered within aerotactic magnetic field intensity range (i.e., from 0 to the minimum magnetic field intensity of magneto-aerotactic magnetic field intensity range). But when the directional magnetic field increases to magnetotactic magnetic field intensity range, the influence of the tumble phase is greatly reduced and during the run phase, the transpotherapeutic complex travels along the magnetic axis in the direction of the magnetic field with no apparent stationary phases caused by tumbles in the hypoxic zones. When the run phase is followed by a reverse phase where transpotherapeutic complexes move backward (maintaining heading towards the direction of the magnetic field for polar magneto-aerotactic-responsive bacteria cells) along the magnetic field axis, since the swimming velocity of polar magneto-aerotactic-responsive bacteria is faster (more efficient) forward than backward due to the propelling force or flagella being located on only on side of the polar magneto-aerotactic-responsive bacteria (and not on both sides like axial magneto-aerotactic-responsive bacteria cells), the net resulting displacement occurs towards the North pole of the magnetic field for North Seeking (NS) magneto-aerotactic-responsive bacteria. (Note that magneto-aerotactic-responsive bacteria can be repolarized to be South Seeking (SS) and vice-versa if required).

The transpotherapeutic complexes when exposed to a directional magnetic field of the magnetic field configurations in the magnetotactic magnetic field intensity range will therefore have run-and-tumble aerotactic searching motions being limited due to the higher magnetic directional torque induced on the chain of magnetosomes in the magneto-aerotactic-responsive bacterium cell. Therefore, a run-and-reverse strategy most likely dominates when exposed to a directional field in the magnetotactic magnetic field intensity range. Magneto-aerotactic-responsive bacteria cells typically exhibit long unidirectional runs being interrupted by short reversal events with a specific mean aperiodic frequency, resulting in forward displacements.

When the transpotherapeutic complexes operate in the aerotactic magnetic field intensity range, the transpotherapeutic complexes in the target oxygen level region will execute run-and-tumble and run-and-reverse motions to maintain their position at this oxygen concentration, forming bands of transpotherapeutic complexes spreading within or close to the targeted hypoxic zones. The volume occupied by the transpotherapeutic complexes inside or in the vicinities of the hypoxic zones corresponds to the OATZ (Oxic-Anoxic Transition Zone). The duration at which the transpotherapeutic complexes will remain in the target oxygen level regions (oxic-anoxic transition zone) such as hypoxic zones are referred to here as TOLRD (Target Oxygen Level Retention Duration) being an average or median duration during which transpotherapeutic complexes will maintain their position in a target oxygen level region such as in a tumor hypoxic zone. For aerotactic magnetic field intensity range, target oxygen level retention duration=1.0 means that the transpotherapeutic complexes will remain in the target oxygen level regions once it reaches the target oxygen level regions until velocity post-injection=0 (velocity post-injection is defined in the next section). For magnetotactic magnetic field intensity range, target oxygen level retention duration=0 means that the transpotherapeutic complexes will cross the target oxygen level regions without apparent retention (caused by no apparent tumbles but only short reverse motions with longer run phases) in the target oxygen level regions. For magneto-aerotactic magnetic field intensity range, target oxygen level retention duration varies from slightly above 0 to slightly below 1.0 from the lower end (MIN-MAFIR) and upper end (MAX-MAFIR) of magneto-aerotactic magnetic field intensity range respectively.

Velocity Post-Injection (VPI)

The Velocity Post-Injection (VPI) is typically characterized by a regression when exposed to physiological environmental factors that can reduce the motility of the magneto-aerotactic-responsive bacterium transpotherapeutic complex. The physiological temperature of 37° C. is one potential factor. The resulting velocity post-injection at a specific time following the injection can be further adjusted by applying an adjustment coefficient to compensate for the impact of a specific physiological or tumoral microenvironment. Velocity post-injection can be expressed as an equation (e.g. velocity post-injection=0.4 (0.1 t²-8.1 t+188) where t is the time after injection expressed in minutes with velocity post-injection in μm^{s-1}), in graphical form, table, etc. Velocity post-injection is the translational velocity, i.e. the velocity calculated from the straight distance travelled between two points. Since a magneto-aerotactic-responsive bacterium cell is typically not passively pushed or pulled by the flagella located on one side of the cell but the cell itself contributes to the displacement efficacy by moving following an helical motion due to an angle between the longitudinal axis of the magnetosomes chain and the axis of the flagellar propelling structures in order to increase the displacement velocity further across fluidic environments, the real or instantaneous velocity of the transpotherapeutic transporters or transpotherapeutic complexes would be related to the total helical distance. Therefore, the instantaneous (transverse or rotational) velocity would be different than the translational (longitudinal) velocity, the latter (translational velocity) being what is really accounted for when estimating the total distance travelled.

Velocity Transpotherapeutic Complex Standard Deviation (VTCSD)

The Velocity of the Transpotherapeutic Complexes (VTC) is defined here as velocity post-injection at t=0 if velocity post-injection=velocity of the transpotherapeutic complexes where velocity of the transpotherapeutic complexes is the velocity recorded prior to shipping from the manufacturing site and VDI is the Velocity During Injection. The Standard Deviation (SD) of velocity of transpotherapeutic complexes referred to here as VTCSD, is a measure of the amount of variation of velocity of the transpotherapeutic complexes. A low velocity transpotherapeutic complex standard deviation indicates that the values of velocity of the transpotherapeutic complexes among the transpotherapeutic complexes to be injected tend to be close to the mean velocity of the transpotherapeutic complexes of the set, while a high velocity transpotherapeutic complex standard deviation indicates that the velocity of the transpotherapeutic complexes are spread out over a wider range.

Total Bolus Escape Time (TBET)

Total Bolus Escape Time refers to the total dissolution time of the injected bolus of transpotherapeutic complexes, i.e. the total time needed for all motile transpotherapeutic complexes to escape the injected bolus. For a given bolus, total bolus escape time is increased with an increased Transpotherapeutic Complexes Concentration (TCC) which results in lower injected volume (VOL) per injection for a given therapeutic dose which may be desirable especially for larger volume while increasing bolus viscosity which improves retention in tissue. For a given transpotherapeutic complex, total bolus escape time is also dependent on the Bolus Escape Surface (BES). Bolus escape surface is defined here as the surface of the injected bolus of transpotherapeutic complexes that is oriented in the North direction of the applied magnetic field configurations for north seeking transpotherapeutic complexes. Transpotherapeutic complexes on the bolus escape surface are generally the first ones to escape the injected bolus. Depending on the geometry of the bolus, bolus escape surface can be constant or variable as the volume of the bolus decreases. Bolus escape surface can therefore be adjusted by shaping the injected bolus appropriately using various injection techniques.

Main Magneto-Aerotactic Targeting Methods (MATM)

Magneto-aerotactic targeting methods can be Single Magneto-Aerotactic Targeting Methods (SMATM) or Combined Magneto-Aerotactic Targeting Methods (CMATM). Single magneto-aerotactic targeting methods include magnetic field intensity range-hypoxic zone targeting, velocity post injection-hypoxic zone targeting, velocity transpotherapeutic complex standard deviation-hypoxic zone targeting, and total bolus escape time-hypoxic zone targeting. Combined magneto-aerotactic targeting methods combine two or more single magneto-aerotactic targeting methods.

Magnetic Field Intensity Range-Hypoxic Zone Targeting

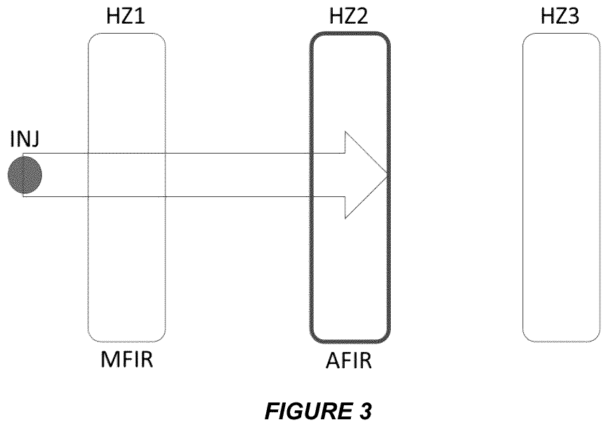

The magnetic field intensity range-hypoxic zone Targeting method modulates the magnetic field intensity range along a displacement path generated by magnetotactic targeting modes or derivatives to target hypoxic zones. A simple example is depicted in FIG. 3. In FIG. 3, hypoxic zone 2 is the target. From the site of injection (INJ) a directional magnetic field configuration represented by the arrow in FIG. 3 is applied. To target hypoxic zone 2, hypoxic zone 1 is bypassed by applying the directional magnetic field configurations in the magnetotactic magnetic field intensity range followed by aerotactic magnetic field intensity range when the transpotherapeutic complexes are sufficiently close or within hypoxic zone 2.

Although magnetic field intensity range-hypoxic zone targeting allows a good control in selecting of the targeted hypoxic zones, it also requires adequate information about the distribution and locations of the hypoxic zones typically previously gathered and estimated from a suitable medical imaging modality, as well as a relatively good estimation of the distance travelled by the transpotherapeutic complexes from injection site with regard to the location of the target hypoxic zones. To take full advantage of magnetic field intensity range-hypoxic zone targeting, velocity transpotherapeutic complex standard deviation are to be relatively low and with a mean value that is sufficiently high to provide sufficient HBPIT (Hypoxic Bypass Post Injection Time). Hypoxic bypass post injection time is defined here as the maximum elapsed time after the injection that the transpotherapeutic complexes will be able to escape hypoxic zones when operating under magnetotactic magnetic field intensity range and it is due mainly to the decay (regression) of velocity post-injection (and hence a decreasing run distance from the hypoxic zones) that occurs particularly in species of magnetotactic bacteria that may be suitable to be used as transpotherapeutic transporters for hypoxic zone targeting. This in turn limits the time that magnetic field intensity range-hypoxic zone targeting can be applied and the total distance from the injection site that the hypoxic zones can be bypassed, although such distance can be relatively high depending on velocity post-injection. Notice also that magnetic field intensity range-hypoxic zone targeting can be used to target various hypoxic zones with various concentrations of transpotherapeutic complexes for instance by targeting hypoxic zone 2 and hypoxic zone 3 with different ratios of transpotherapeutic complexes which could be achieved in the example depicted in FIG. 3 by setting magnetotactic magnetic field intensity range for hypoxic zone 1, magneto-aerotactic magnetic field intensity range for hypoxic zone 2 and aerotactic magnetic field intensity range for hypoxic zone 3. This is just a simple example and other scenarios are also possible.

Velocity Post Injection-Hypoxic Zone Targeting

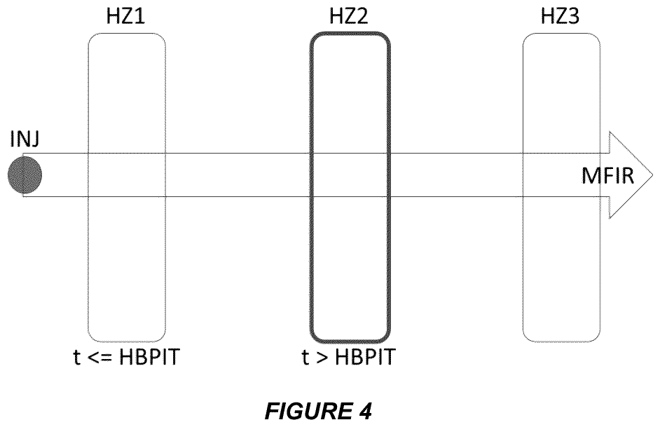

The Velocity Post Injection-Hypoxic Zone Targeting (VPI-HZT) method typically operates in magnetotactic magnetic field intensity range (although magneto-aerotactic magnetic field intensity range could also be used) but relies on the decay of velocity post-injection (velocity post-injection regression) to target hypoxic zones. This is simply illustrated in FIG. 4 where t is the elapsed time following the injection.

In the simple example depicted in FIG. 4, the transpotherapeutic complexes have a sufficiently high velocity post-injection to bypass hypoxic zone 1 with a directional magnetic field configuration in the magnetotactic magnetic field intensity range. When the transpotherapeutic complexes continue travelling along the displacement path resulting from the magnetic field configuration, the decayed velocity post-injection continues to decrease until a value that is no more sufficient to overshoot the hypoxic zones is reached. At this stage, the distance in the run state resulting from the decayed velocity post-injection is insufficient to not be influenced by the oxygen gradient related to the hypoxic zones. Therefore, the transpotherapeutic complexes remain in a specific hypoxic zone represented by hypoxic zone 2 in the example depicted in FIG. 4.

If a particular hypoxic zone is targeted, velocity post injection-hypoxic zone targeting requires a good estimation of the regressed velocity post-injection and the corresponding travelled distance along displacement path. Since the velocity post injection-hypoxic zone targeting method is very dependent on the regressed velocity post-injection, it limits the range of control that can be applied by the operator. Furthermore, the targeting range from the injection site is limited and dictated by the regressed velocity post-injection.

Velocity Transpotherapeutic Complex Standard Deviation-Hypoxic Zone Targeting

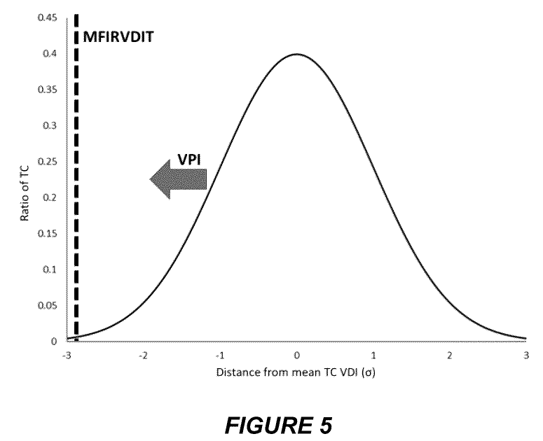

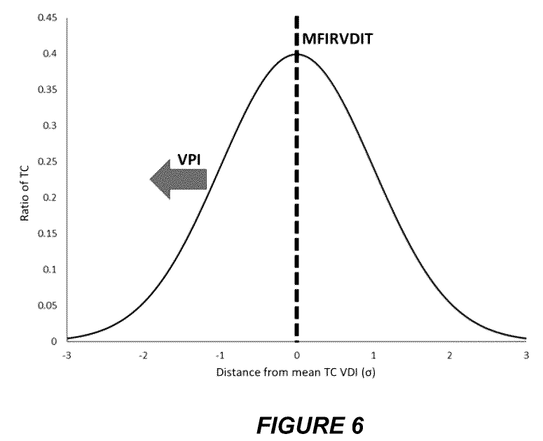

The Velocity Transpotherapeutic Complex Standard Deviation-Hypoxic Zone Targeting (VTCSD-HZT) method exploits the distribution of transpotherapeutic complexes velocity of the transpotherapeutic complexes and therefore the velocity post-injection variations in the injected volume to target hypoxic zones. The fundamental concept behind the velocity transpotherapeutic complex standard deviation-hypoxic zone targeting method is depicted in FIG. 5 and FIG. 6 and depends mainly on velocity transpotherapeutic complex standard deviation, the mean transpotherapeutic complex velocity during injection, and the regression rate of velocity during injection indicated by the left-pointing arrow in FIG. 5 and FIG. 6.

FIG. 5 shows an example of velocity transpotherapeutic complex standard deviation of a batch of transpotherapeutic complexes measured at the factory. In this example, only the fastest transpotherapeutic complexes have been selected by rejecting the slowest transpotherapeutic complexes. Assuming that velocity during injection which is velocity post-injection at t=0 is the same as velocity of the transpotherapeutic complexes, then the first hypoxic zones closer to the injection site will likely be bypassed by the transpotherapeutic complexes if operating in magnetotactic magnetic field intensity range since all the population of transpotherapeutic complexes velocity during injection and velocity post-injection shortly after injection are faster than magnetotactic magnetic field intensity range velocity during injection threshold defined here as the minimum velocity during injection required to bypass hypoxic zones. As the time following injection elapses, velocity post-injection reduces while maintaining magnetotactic magnetic field intensity range, contributing to target hypoxic zones. The rate and distribution of hypoxic zone targeting in hypoxic zones along displacement path will depend on velocity transpotherapeutic complex standard deviation with a high velocity transpotherapeutic complex standard deviation providing hypoxic zone targeting to increase the spreading of the magneto-aerotactic-responsive bacteria throughout the hypoxic zones along the displacement path and less transpotherapeutic complexes density per hypoxic zone, while a low velocity transpotherapeutic complex standard deviation would result in more localized (less spread) hypoxic zones but with higher density of transpotherapeutic complexes per hypoxic zone.

FIG. 6 depicts a magnetotactic magnetic field intensity range velocity during injection threshold that is located at the mean transpotherapeutic complexes velocity during injection, allowing hypoxic zones closer to the injection site as well as other hypoxic zones located further away from the injection site to be targeted while maintaining the magnetotactic magnetic field intensity range. Hence, batches of the same transpotherapeutic complexes can be designed to have different ratios of percentages of transpotherapeutic complexes populations that are left or right of magnetotactic magnetic field intensity range velocity during injection threshold in the velocity transpotherapeutic complex standard deviation to optimize hypoxic zone targeting with regard to hypoxic zones distribution and locations in the tumor volume, this approach being the fundamental idea behind the velocity transpotherapeutic complex standard deviation-hypoxic zone targeting method. In general, to target most hypoxic zones that may be relatively far apart, a high velocity transpotherapeutic complex standard deviation indicating that the velocity of the transpotherapeutic complexes are spread out over a wider range of velocities may be more suitable when relying on the velocity transpotherapeutic complex standard deviation-hypoxic zone targeting method only.

Total Bolus Escape Time-Hypoxic Zone Targeting

The Total Bolus Escape Time-Hypoxic Zone Targeting (TBET-HZT) method relies on the dissolution rate of the injected transpotherapeutic complexes bolus combined with velocity post-injection regression to achieve hypoxic zone targeting in the tumor volume with a directional vector typically operating in magnetotactic magnetic field intensity range (although magneto-aerotactic magnetic field intensity range could also be used). Considering the Surface-to-Volume (SN) ratio of a spherical injected bolus where surface=12.57 r² and volume=4.189 r³, r being the radius of the spherical bolus, we can conclude that by dividing the single injected bolus as smaller injected boluses would lead to a larger effective bolus escape surface and therefore reduce total bolus escape time. The same idea applies by modifying the volumetric shape of the bolus where a cylindrical-shaped bolus for instance with the longitudinal axis oriented at right-angle from directional vector will yield a higher surface-to-volume ratio leading to a shorter total bolus escape time. Typically, total bolus escape time is equal to or shorter than magnetotactic magnetic field intensity range velocity during injection threshold to target hypoxic zones in the tumor volume beyond the injection site when a magnetic field configuration in magnetotactic magnetic field intensity range is applied since a total bolus escape time greater than magnetotactic magnetic field intensity range velocity during injection threshold will result in a percentage of transpotherapeutic complexes populations remaining at the injection site which might also be suitable in particular cases. Besides bolus escape surface, other factors may also influence and be used to adjust total bolus escape time, transpotherapeutic complexes concentration being one example.

As for the transpotherapeutic complexes at the bolus escape surface escaping the bolus and travelling towards hypoxic zones, the transpotherapeutic complexes in the bolus are also exposed to physiological environmental factors that can reduce the motility of the transpotherapeutic complexes such that as the time after the injection elapses, the regressed velocity during injection for the transpotherapeutic complexes escaping the bolus will decrease such that the last transpotherapeutic complexes escaping the bolus will have a velocity during injection that is lower than the velocity during injection of the first portion of transpotherapeutic complexes that escaped the bolus.

If total bolus escape time=motility Time with directional vector in the magnetotactic magnetic field intensity range and a mean velocity during injection sufficiently higher than magnetotactic magnetic field intensity range velocity during injection threshold with a very low velocity transpotherapeutic complex standard deviation, then we can expect that the first transpotherapeutic complexes to escape the injected bolus will bypass the first hypoxic zones and target hypoxic zones further way from the injection site when the velocity post-injection will regress below the magnetotactic magnetic field intensity range velocity during injection threshold. The following transpotherapeutic complexes that escaped the injected bolus will then reach the hypoxic zones at a shorter distance from the injection site and therefore target hypoxic zones closer to the injection site while the last transpotherapeutic complexes escaping the injected bolus will not have sufficient velocity post-injection to escape the closest hypoxic zones from the injection site resulting to the closest hypoxic zones from the injection site to be targeted as well. If total bolus escape time is adjusted to be much shorter, hypoxic zone targeting closer to the injection site might not occur while increasing hypoxic zone targeting further away from the injection site.

Combined Magneto-Aerotactic Targeting Methods

Combined Magneto-Aerotactic Targeting Methods (CMATM) combines two or more of the single magneto-aerotactic targeting methods described in previous sections.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

What is claimed is:

1. A method of improving targeted delivery of at least one of a treatment agent, an imaging agent and a diagnostic agent attached to magneto-aerotactic-responsive bacteria adapted to self-locomote while subject to a magnetic field, the magneto-aerotactic-responsive bacteria depositing the at least one of a treatment agent, an imaging agent and a diagnostic agent at hypoxic zones in a patient, comprising:
adapting a total bolus escape time of a solution of the magneto-aerotactic responsive bacteria when injected into the patient in order to influence the targeting of a target zone with hypoxic zones in the patient, wherein decreasing the total bolus escape time favors a targeting, by the magneto-aerotactic-responsive bacteria, of one or more of the hypoxic zones at a farther distance from an injection site of the magneto-aerotactic-responsive bacteria while considering a decrease in velocity post-injection of said magneto-aerotactic-responsive bacteria when exposed to an environment of said patient.

2. The method as defined in claim 1, wherein the total bolus escape time is adapted by adjusting one or more of:
a concentration of the magneto-aerotactic-responsive bacteria in the solution;
a volume of the bolus;
a viscosity of the solution; and
a surface of the bolus exposed to a north of the magnetic field when the magneto-aerotactic-responsive bacteria are north-seeking, or a surface of the bolus exposed to a south of the magnetic field when the magneto-aerotactic-responsive bacteria are south-seeking.

3. The method as defined in claim 1, wherein the total bolus escape time is adapted by dividing, during injection, the bolus into smaller boluses injected into the patient in order to lower a bolus-volume as the individual volumes of each of the smaller boluses is less than the volume of the bolus.

4. The method as defined in claim 1, wherein increasing the total bolus escape time is achieved by heating the solution in the injection device prior to introduction into the patient.

5. The method as defined in claim 1, wherein increasing the total bolus escape time is achieved by reducing the rate of volume of injection into the patient.

6. The method as defined in claim 1, wherein the total bolus escape time is adapted by adjusting the shape of the bolus.

7. The method as defined in claim 1, wherein decreasing the total bolus escape time is in order to enable access to a portion of a tumor that is inaccessible by the injection device, necessitating the travel of the magneto-aerotactic-responsive bacteria from the injection point to the portion of the tumor.

8. The method as defined in claim 1, wherein the total bolus escape time is adapted by influencing a shape of the bolus through a selection of an appropriate injector.

9. The method as defined in claim 1, wherein the total bolus escape time is decreased by increasing the surface to volume ratio of the injected bolus.

10. The method as defined in claim 1, wherein an increase in total bolus escape time is favoured when targeting of hypoxic zones closer to the injection site is sought.

11. A method of influencing a total bolus escape time of a solution of magneto-aerotactic-responsive bacteria attached to at least one of a treatment agent, an imaging agent and a diagnostic agent for injection into a patient, wherein the magneto-aerotactic-responsive bacteria is adapted to self-locomote while subject to a magnetic field, the magneto-aerotactic-responsive bacteria depositing the at least one of a treatment agent, an imaging agent and a diagnostic agent at a target zone with hypoxic zones in a patient, comprising:
at least one of increasing the surface-volume ratio of the bolus, decreasing the concentration of the magneto-aerotactic-responsive bacteria in the solution, decreasing the viscosity of the solution, and increasing the surface of the bolus exposed to the north of the magnetic field for north-seeking magneto-aerotactic-responsive bacteria and to the south of the magnetic field for south-seeking magneto-aerotactic-responsive bacteria, that is to decrease the total bolus escape time of the magneto-aerotactic-responsive bacteria; or
at least one of decreasing the surface-volume ratio of the bolus, increasing the concentration of the magneto-aerotactic-responsive bacteria in the solution, increasing the viscosity of the solution, and decreasing the surface of the bolus exposed to the north of the magnetic field for north-seeking magneto-aerotactic-responsive bacteria and to the south of the magnetic field for south-seeking magneto-aerotactic-responsive bacteria, that is to increase the total bolus escape time of the magneto-aerotactic-responsive bacteria,
whereby a lower total bolus escape time of the magneto-aerotactic responsive bacteria is for targeting one or more of the hypoxic zones of the target zone in said patient that are further from said injection site.

12. The method as defined in claim 11, whereby targeting of the one or more of the hypoxic zones in a tumor further from said injection site is considered when the injection device cannot access a region next to said hypoxic zones, requiring a greater travel of the magneto-aerotactic-responsive bacteria from the injection site to the hypoxic zones, the method comprising at least one of increasing the surface-volume ratio of the bolus, decreasing the concentration of the magneto-aerotactic-responsive bacteria in the bolus, decreasing the viscosity of the bolus, and increasing the surface of the bolus exposed to the north of the magnetic field for north-seeking magneto-aerotactic-responsive bacteria and to the south of the magnetic field for south-seeking magneto-aerotactic-responsive bacteria to decrease the total bolus escape time of the magneto-aerotactic-responsive bacteria.

13. The method as defined in claim 11, comprising at least one of increasing the surface-volume ratio of the bolus by dividing the bolus into smaller boluses.

14. The method as defined in claim 11, wherein the magneto-aerotactic responsive bacteria are attached to a treatment agent.

15. The method as defined in claim 11, wherein the bolus is prepared by further considering the velocity distribution of the magneto-aerotactic responsive bacteria in the bolus.
